(19) United States
(12) Patent Application Publication
(10) Pub. No.: US 2019/0106454 A1
(43) Pub. Date: Apr. 11, 2019

(54) COMPOUNDS LINKED WITH A SACCHARIDE METAL COMPLEX AND USES THEREOF

(71) Applicant: FRED HUTCHINSON CANCER RESEARCH CENTER, sEATTLE, WA (US)

(72) Inventors: Richard A. ZAGER, Seattle, WA (US); Julian SIMON, Seattle, WA (US)

(21) Appl. No.: 16/086,987

(22) PCT Filed: Mar. 23, 2017
(86) PCT No.: PCT/US17/23883

§ 371 Date: Sep. 20, 2018

Related U.S. Application Data
(60) Provisional application No. 62/312,358, filed on Mar. 23, 2016.
(60) Provisional application No. 62/456,001, filed on Feb. 7, 2017.

(51) Int. Cl. C07H 23/00; A61K 47/54

(52) U.S. Cl. CPC C07H 23/00; A61K 47/546; A61K 47/551; A61K 47/549; A61K 47/547

(57) ABSTRACT

The present disclosure provides compounds linked with a saccharide-metal complex. The compounds are designed to protect organs by inducing acquired cytoresistance without causing injury to the organ, among other potential uses.

### CROSS-REFERENCE TO RELATED APPLICATIONS

This application claims priority to U.S. Provisional Patent Application No. 62/456,001 filed on Feb. 7, 2017, and U.S. Provisional Patent Application No. 62/312,358 filed on Mar. 23, 2016. The present disclosure is related to that described in Patent Cooperation Treaty Application No. PCT/US2015/052676, filed on Sep. 28, 2015, U.S. Provisional Patent Application No. 62/057,047 filed Sep. 29, 2014, and U.S. Provisional Patent Application No. 62/212,232 filed Aug. 31, 2015. The entire contents of each of these five documents are speficially incorporated by reference herein.

### STATEMENT REGARDING FEDERALLY SPONSORED RESEARCH OR DEVELOPMENT

This invention was made with government support under grant DK038432 awarded by the National Institutes of Health. The government has certain rights in the invention.

### FIELD OF THE DISCLOSURE

The present disclosure provides formulations and kits including compounds linked with a saccharide-metal complex and methods of using the same

### BACKGROUND OF THE DISCLOSURE

Tetrapyrrole-based compounds are found throughout nature and can be involved in numerous biochemical processes. Tetrapyrroles can include four pyrrole rings coupled to each other via covalent bonding. In some cases, the pyrrole rings can be joined by a carbon bridge (e.g., a single carbon CH or CH₂ bridge). The pyrrole rings of tetrapyrrole compounds can be arranged in a cyclic configuration. When the pyrrole rings of tetrapyrrole compounds are arranged in a cyclic configuration, the nitrogen atoms in pyrrole rings can chelate a metal.

Saccharides are molecules including carbon atoms, oxygen atoms, and hydrogen atoms arranged as aldehydes or ketones with additional hydroxyl groups. Saccharides can be configured in a chain structure or a heterocyclic structure. In some cases, monosaccharides can include aldehyde compounds or ketone compounds that have five or six carbons atoms and at least two hydroxyl groups coupled to the carbon atoms, such as glucose, fructose, and galactose. Monosaccharides can be joined via covalent bonding to produce disaccharides. In an example, joining a glucose molecule with a fructose molecule produces the disaccharide sucrose. In another example, joining two glucose molecules produces the disaccharide maltose. In an additional example, joining a galactose molecule and a glucose molecule produces the disaccharide lactose. In some cases, monosaccharides, disaccharides, or combinations thereof, can form complexes with a metal.

### SUMMARY OF THE DISCLOSURE

The current disclosure provides formulations including compounds linked with saccharide-metal complexes. In particular, tetrapyrrole compounds can be linked with the saccharide-metal complexes. The tetrapyrrole compounds can include porphyrin compounds. In some examples, the tetrapyrrole compounds can include porphyrin. In particular embodiments, the tetrapyrrole compounds can include a protoporphyrin. Also, the tetrapyrrole compounds can include a mesoporphyrin. In addition, the tetrapyrrole compounds can include vitamin B12, also referred to herein as cobalamins. In particular embodiments, the saccharide-metal complex can include an iron-sucrose complex.

The linking components used to join the tetrapyrrole compounds and the saccharide-metal complexes can form relatively labile bonds between the tetrapyrrole compounds and the saccharide-metal complexes. For example, the linking components can form ester linkages between the tetrapyrrole compounds and the saccharide-metal complexes. In other examples, the linking components can form hydrazone linkages between the tetrapyrrole compounds and the saccharide-metal complexes. In particular embodiments, the linking components used to join the tetrapyrrole compounds and the saccharide-metal complexes can form relatively non-labile bonds between the tetrapyrrole compounds and the saccharide-metal complexes. To illustrate, the linking components can form amide linkages between the tetrapyrrole compounds and the saccharide-metal complexes.

The tetrapyrrole compounds can be linked with a saccharide-metal complex to produce a formulation that can be administered to protect an organ without causing injury to the organ. Additionally, the formulation can be administered to generate acquired cytoresistance in an organ without causing injury to the organ. Further, the formulation can be administered to up-regulate expression of protective stress proteins in an organ without causing injury to the organ. In particular embodiments, the molecule formed by linking a saccharide-metal complex with a tetrapyrrole compound can be cleaved upon being administered to minimize or eliminate injury to an organ.

### BRIEF DESCRIPTION OF THE FIGURES

FIG. 1 includes a process directed to making and using formulations including compounds linked with a saccharide-metal complex.

FIG. 2 and FIG. 3 show that vitamin B12 and iron-sucrose each induces marked HO-1 protein increases within 4 hrs and persists for 18 hrs of their IV injection.

FIG. 4 shows maleate injection caused severe AKI as denoted by marked BUN and PCr increases over maleate injected controls (C). Neither Sn-protoporphyrin alone nor iron-sucrose alone significantly altered the severity of renal injury. However, combined iron-sucrose+Sn-protoporphyrin conferred marked protection, as denoted by 75% reductions in BUN/PCr concentrations (the horizontal lines represent the means of BUN/PCr levels in normal mice).

FIG. 5 shows that within 18 hrs of inducing IRI, 4 fold elevations in BUN and PCr concentrations resulted. Pre-treatment with iron-sucrose+Sn-protoporphyrin conferred significant protection, lowering the BUN and PCr levels by 50%. The horizontal lines represent mean BUN/PCr levels in normal mice.

FIG. 6 shows that maleate injection induced severe AKI. Pre-treatment with iron-sucrose+B12 markedly mitigated this injury, as denoted by BUN/PCr reductions. The horizontal lines represent mean BUN/PCr levels in normal mice.

FIG. 7 shows severe renal failure resulted within 18 hrs of glycerol injection. Pre-treatment with iron-sucrose+B12 conferred substantial functional protection, as denoted by marked reductions in both 18 hr BUN and PCr concentrations. The horizontal lines represent mean BUN/PCr levels for normal mice.

FIG. 8 shows marked and significant increases in HO-1 mRNA, as assessed 4 hr post injection. By 18 hrs, HO-1 mRNA levels returned to normal values.

FIG. 9 shows the 4 hr mRNA increases are correlated with a significant increase in HO-1 protein levels. These levels remained elevated at the 18 hr time point, particularly in the case of iron-sucrose administration.

FIG. 10 shows Ferrtin heavy chain Western blotting of renal cortex obtained from normal mice, and from mice 18 hrs after either SnPP, FeS, or SnPP+FeS injection.

FIG. 11. Comparison of SnPP vs. SnMeP effects on renal cortical HO-1 and haptoglobin mRNA induction 4 hrs post agent injection. Mice received either 1 umole tin protoporphyrin (SnPP), tin mesoporphyrin (SnMeP), or vehicle (n, 3 each) and 4 hrs renal cortical RNA was extracted and subjected to RT-PCR. Both agents raised HO-1 and haptoglobin mRNA and to comparable degrees, suggesting equivalent biologic effects.

FIG. 12. Tin mesoporphryin effects on renal cortical and plasma HO-1 protein levels at 4 and 18 hrs post 1 umole injection. By 4 hrs and 18 hrs post Sn mesoporphyrin injection, 3 fold and 5 fold increases in renal cortical HO-1 levels over baseline (BL) were observed, as detected by ELISA. Plasma HO-1 levels rose 15 fold by 18 hrs post injection (n, 3-4 per group).

FIG. 13. Tin mesoporphryin effects on renal cortical and plasma HO-1 protein levels at 4 and 18 hrs post 1 umole injection. At 4 and 18 hrs post Sn mesoporphyrin injection, stepwise increases in renal cortical and plasma haptoglobin levels were observed (n, 3-4 per group).

FIG. 14. Tin mesoporphyrin pre-treatment mitigates renal ischemic injury. Mice received either 1 umole of Sn mesoporphyrin (MeP; n, 3) or vehicle (n, 4) and 18 hrs later they were all subjected to 22 min of bilateral ischemic injury. MeP pretreatment induced a lessening of ischemic damage, as reflected by reductions in BUN and plasma creatinine concentrations.

### DETAILED DESCRIPTION

The current disclosure provides formulations including compounds linked with saccharide-metal complexes. In particular, tetrapyrrole compounds can be linked with the saccharide-metal complexes. The tetrapyrrole compounds can include porphyrin compounds. In some examples, the tetrapyrrole compounds can include porphyrin. In particular embodiments, the tetrapyrrole compounds can include a protoporphyrin. Also, the tetrapyrrole compounds can include a mesoporphyrin. In addition, the tetrapyrrole compounds can include vitamin B12, also referred to herein as cobalamins. In particular embodiments, the saccharide-metal complex can include an iron-sucrose complex.

The linking components used to join the tetrapyrrole compounds and the saccharide-metal complexes can form relatively labile bonds between the tetrapyrrole compounds and the saccharide-metal complexes. For example, the linking components can form ester linkages between the tetrapyrrole compounds and the saccharide-metal complexes. In other examples, the linking components can form hydrazone linkages between the tetrapyrrole compounds and the saccharide-metal complexes. In particular embodiments, the linking components used to join the tetrapyrrole compounds and the saccharide-metal complexes can form relatively non-labile bonds between the tetrapyrrole compounds and the saccharide-metal complexes. To illustrate, the linking components can form amide linkages between the tetrapyrrole compounds and the saccharide-metal complexes.

The tetrapyrrole compounds can be linked with a saccharide-metal complex to produce a formulation that can be administered to protect an organ without causing injury to the organ. Additionally, the formulation can be administered to generate acquired cytoresistance in an organ without causing injury to the organ. Further, the formulation can be administered to up-regulate expression of protective stress proteins in an organ without causing injury to the organ. In particular embodiments, the molecule formed by linking a saccharide-metal complex with a tetrapyrrole compound can be cleaved upon being administered to minimize or eliminate injury to an organ.

The disclosed compounds were designed for use in “acquired cytoresistance.” Injury to a bodily organ can elicit protective responses by the organ such that it is able to better protect itself should injurious events continue or re-occur. This protective phenomenon is known in the art as “ischemic preconditioning” or “acquired cytoresistance.”

The current disclosure provides formulations, kits, and methods that allow the induction of acquired cytoresistance without injury to the organ that is to be protected. The formulations, kits, and methods can be used in a clinical setting to preemptively protect organs without causing injury to the organ because of the acquired cytoresistance that can be induced using the formulations, kits, and methods described herein. In some cases, administering the formulations described herein can take place when a known insult is approaching. Without being bound by theory, the formulations, kits, and methods described herein induce acquired cytoresistance by up-regulating expression of protective stress proteins. In particular embodiments, induction of acquired cytoresistance can also be achieved by administering compounds that up-regulate stress proteins through the same or similar biological pathways utilized by heme proteins.

As shown in the experimental examples described herein, metal-saccharide complexes (e.g., iron-sucrose) and porphyrin-based molecules have a synergistic effect in the protection of organs from injury. The implementations described herein build off of those results by producing conjugates that link a metal-saccharide complex with a porphyrin-based molecule. The use of a conjuage including a metal-saccharide complex linked with a porphyrin-based molecule can simplify the delivery of the synergistic compounds to a subject.

An “insult” is an occurrence that is likely to cause injury to an organ. Example insults include shock (low blood pressure), kidney hypoperfusion, surgery, induced cardiac or cerebral ischemic-reperfusion, cardiopulmonary bypass, balloon angioplasty, radiocontrast toxicity administrations, chemotherapy, drug administration, nephrotoxic drug administration, blunt force trauma, puncture, poison, smoking, etc. In the context of storing organs for transplantation, an insult can include ischemia reperfusion or cold storage.

An “injury” is a detrimental effect on an organ evidenced by cell death within the organ, cell damage within the organ, damaged structure within the organ and/or decreased function of the organ as compared to one or more relevant control groups, conditions, or reference levels.

“Absence of injury” to an organ, “without causing an injury” to an organ, “does not injure the organ” and similar phrases mean that any effect on an organ is, within the scope of sound medical judgment, commensurate with a reasonable benefit/risk ratio of administration. In particular embodiments, absence of an injury can be demonstrated by showing that the function of an organ is not statistically significantly different from a relevant control group, condition, or reference level according to a known test of organ function at the time using appropriate statistical comparisons. Example assays of organ function include measuring markers associated with organ function; measuring the output of an organ; and measuring a performance metric of the organ as compared to one or more relevant control groups, conditions or reference levels.

“Protecting an organ from injury” and similar phrases include one or more of: up-regulating the expression of protective stress proteins; preserving organ function in whole or in part (e.g., measuring the output of an organ; measuring a performance metric of the organ); reducing organ cell injury (in particular embodiments, as manifested by decreased leakage of intracellular proteins into the circulation), and reducing cell death within the organ as compared to one or more relevant control groups, conditions, or reference levels.

Numerous assays that can be used to assess presence or absence of an injury and associated protection are disclosed herein and can be used in animal and human models of organ function. Lack of injury and/or protection of an organ can be confirmed by comparing a relevant measure from a subject with a reference level. Reference levels can include “normal” or “control” levels or values, defined according to, e.g., discrimination limits or risk defining thresholds, in order to define cut-off points and/or abnormal values for organ function. The reference level can be a level of an indicia typically found in a subject who is not suffering from organ injury. Other terms for “reference levels” include “index,” “baseline,” “standard,” “healthy,” “pre-injury,” etc. Such normal levels can vary, based on whether some indicia are used alone or in a formula combined with other indicia to output a score. Alternatively, the reference level can be derived from a database of scores from previously tested subjects who did not develop organ injury over a clinically relevant time period. Reference levels can also be derived from, e.g., a control subject or population whose organ injury status is known. In particular embodiments, the reference level can be derived from one or more subjects who have been exposed to treatment for an organ injury, or from subjects who have shown improvements in organ function following injury as a result of exposure to treatment. In particular embodiments, the reference level can be derived from one or more subjects with organ injury who have not been exposed to treatment. A reference level can also be derived from injury severity algorithms or computed indices from population studies.

In particular embodiments, a “reference level” can refer to a standardized value for organ function which represents a level not associated with any injury; a level associated with a particular type of injury; a level associated with a severity of injury; or a level associated with a particular subject at the time of a diagnosis, at the beginning of a treatment, or at a time point during a treatment. The reference level can be a universal reference level which is useful across a variety of testing locations or can be a reference level specific for a testing location and specific assay used to measure the organ function. In particular embodiments, the reference level is derived from (i) an individual who does not have organ injury or organ injury of a particular type; or (ii) a group of individuals who do not have organ injury or organ injury of a particular type. Reference levels for a subject can also be related to time points of the subject undergoing treatments to monitor the natural progression or regression of organ injury in the subject.

In particular embodiments, reference levels can be derived from a “dataset”. A dataset represents a set of numerical values resulting from evaluation of a sample (or population of samples) under a desired condition. The values of the dataset can be obtained, for example, by experimentally obtaining measures from a sample and constructing a dataset from these measurements; or alternatively, by obtaining a dataset from a service provider such as a laboratory, or from a database or a server on which the dataset has been stored.

Without being bound by theory, the up-regulation of a number of stress proteins leads to the induction of acquired cytoresistance. “Up-regulation” includes an increase in expression of a gene or nucleic acid molecule of interest or the activity of a protein, e.g., an increase in gene expression or protein activity as compared to the expression or activity in an otherwise identical or comparable gene or protein that has not been up-regulated.

Up-regulation of the following example stress proteins can lead to the induction of acquired cytoresistance: heme oxygenase (HO), haptoglobin, hemopexin, hepcidin, alpha-1 antitrypsin (AAT), interleukin-10 (IL-10), heat-shock proteins, neutrophil gelatinase-associated lipocalin (NGAL), and HMG CoA reductase.

Expression of protective stress proteins is up-regulated by administration of heme proteins. Heme proteins are metalloproteins that contain a heme prosthetic group (e.g., a protoporphyrin ring with a central Fe). A protoporphyrin ring includes four pyrrole rings linked by methine bridges. Four methyl, two vinyl, and two propionate side chains can also be attached to the pyrrole rings. In particular embodiments, the heme proteins are low molecular weight heme proteins. “Low molecular weight heme proteins” include those with a molecular weight of 35 kDa or less, 33, kDa or less, 31 kDa or less, 30 kDa or less, 28 kDa or less, 26 kDa or less, 25 kDa or less; 24 KDa or less; 23 kDa or less; 22 kDa or less; 21 kDa or less; 20 kDa or less; 19 kDa or less; 18 kDa or less; 17 kDa or less; 16 kDa or less; 15 kDa or less; 14 kDa or less; 13 kDa or less; 12 kDa or less; 11 kDa or less; or 10 kDa or less. In particular embodiments, heme proteins are rapidly cleared when administered intravenously. “Rapidly cleared” means a urinary excretion rate that has a urinary clearance rate of >50% of serum creatinine or urea. In particular embodiments the heme proteins are low molecular weight heme proteins that, when administered intravenously, are cleared at a rate equal to or lower than the rate at which creatine or urea are cleared. Myoglobin is one heme protein that can be used with the formulations, kits, and methods disclosed herein. References to heme proteins include modified heme proteins, variant heme proteins and D-substituted analog heme proteins. References to myoglobin include modified myoglobin, variant myoglobin and D-substituted analog myoglobin.

Approaches that allow heme protein administration to induce acquired cytoresistance without causing an organ injury include selecting a therapeutically effective amount of the heme protein; increasing the biological half-life of the heme protein; potentiating the action of the heme protein; and reducing toxicity associated with heme protein administration.

The biological half-life of a heme protein can be extended by administering the heme protein in combination with a heme protein degradation inhibitor. In particular embodiments, heme protein degradation inhibitors can reduce or eliminate the cleavage of the heme protein's porphyrin ring by HO, reducing or eliminating release of the heme protein's toxic Fe content. In particular embodiments, administration of a heme protein degradation inhibitor in combination with a heme protein can allow for administration of lower doses of the heme protein.

In particular embodiments, the heme protein can be modified. The modified protein can include a nitrited heme protein or nitrited heme protein degradation inhibitor. Nitrite is involved in regulating production of nitric oxide (NO) from nitric oxide synthase (NOS) independent pathways. Inorganic nitrite can undergo a one electron reduction back to NO through various mechanisms with oxygen-binding heme proteins (hemoglobin and myoglobin), deoxyhemoglobin, deoxymyoglobin, xanthine oxidoreductase, endothelial NOS, acidic disproportionation, and members of the mitochondrial electron transport chain, e.g., mitochondrial heme proteins all being potential electron donors. In some cases, a metal-saccharide can also be nitrited. In additional examples, a protoporphyrin compound can be nitrited. In further examples, a cobalamin can be nitrited.

Nitrite binding to heme iron, such as in myoglobin, can increase the heme protein's ability to up-regulate expression of stress proteins, such as, heat shock proteins (e.g., HSP 72); HO-1; haptoglobin; hemopexin, hepcidin, IL-10, AAT, NGAL and/or HMG CoA reductase. Nitrite—Fe binding disclosed herein can also decrease toxicity associated with heme protein administration. Without being bound by theory, up-regulated expression of stress proteins serves to promote acquired cytoresistance.

In particular embodiments, the modified protein can include a PEGylated heme protein or heme protein degradation inhibitor. PEGylation is one method that can be used to increase the size of myoglobin and other low molecular weight proteins. PEGylation is a process by which polyethylene glycol (PEG) polymer chains are covalently conjugated to other molecules such as drugs or proteins. Several methods of PEGylating proteins have been reported in the literature. For example, N-hydroxy succinimide (NHS)-PEG was used to PEGylate the free amine groups of lysine residues and N-terminus of proteins; PEGs bearing aldehyde groups have been used to PEGylate the amino-termini of proteins in the presence of a reducing reagent; PEGs with maleimide functional groups have been used for selectively PEGylating the free thiol groups of cysteine residues in proteins; and site-specific PEGylation of acetyl-phenylalanine residues can be performed.

In particular embodiments, any compound that blocks binding of heme to HO can function as a heme protein degradation inhibitor. For example, a number of synthetic analogs of iron protoporphyrin IX are known. These compounds are commercially available and/or can be readily synthesized by known methods. They include, for example, platinum, zinc, nickel, cobalt, copper, silver, manganese, chromium, and tin protoporphyrin IX. For convenience, these compounds can sometimes herein be referred to generically as Me-protoporphyrin or MePP, where Me stands for metal, and specifically by utilizing the chemical symbol for the metal such as Cr-protoporphyrin (CrPP), Sn-protoporphyrin (SnPP), Zn-protoporphyrin (ZnPP) for the chromium, tin, and zinc protoporphyrin compounds respectively.

That blocking the action of HO could beneficially assist in the induction of acquired cytoresistance without causing an injury was unexpected at the time of this finding. For example, Nath et al., showed that knocking out the HO-1 gene in mice worsened renal injury in the glycerol model of heme protein toxicity. The authors stated that HO-1 is a critical protectant against acute heme protein-induced toxicity in vivo. Am. J. of Path., 2000 May 156(5): 1527-1535.

Moreover, that Me-porphyrins could be used in combination with a heme protein to induce acquired cytoresistance without causing an injury was also unexpected. This is because Me-porphyrins were generally thought to adversely affect organs in various models of organ injury. For example, Agarwal et al. found that pretreatment with Sn-protoporphyrin exacerbated renal injury in a HO-based in vivo model of heme protein mediated renal injury. Particularly, pretreatment with Sn-protoporphyrin led to higher serum creatinine values on days 3 through 5 and lower inulin clearances on day 5. Renal hemodynamics studied at day 2 after cisplatin demonstrated reduced renal blood flow rates, increased renal vascular resistance and increased fractional excretion of sodium in rats treated with Sn-protoporphyrin. Kidney Int. 1995 October 48(4): 1298-307. In the glycerol model rhabdomyolysis, Nath et al., found that the kidney responds to high amounts of heme proteins by inducing HO and that blocking the action of HO with a competitive inhibitor (here, Sn-protoporphyrin) exacerbated kidney dysfunction. J. Clin. Invest. 1992 July: 90(1): 267-70. Ferenbach et al., and Goodman et al., have similarly shown that inhibition of HO using Me-protoporphyrins worsens renal damage. See Nephron. Exp. Nephrol. 2010 April 115(3): e33-7 and Kidney Int. 2007 October 72(8): 945-53 respectively. Based on these teachings of the art, one of ordinary skill in the art would not have expected the beneficial effects of HO-1 inhibition currently disclosed.

Without being bound by theory, heme proteins activate redox sensitive transcription factors, leading to the up-regulation of redox sensitive cytoprotective proteins. This pathway is initiated by the iron content of myoglobin. Thus, as demonstrated herein, alternative approaches for inducing iron-mediated renal tubular cytoprotective gene signaling are also effective. These alternative approaches include administration of iron and/or vitamin B12. The rationale for B12 is that both cobalt and cyanide can independently induce HO-1. Thus, B12 represents a safe method to administer both cyanide and cobalt as a single agent, as both are integral parts of the B12 molecule.

In particular embodiments, tin salts can be administered in conjunction with a source of iron. For example, SnCl₂ and iron sucrose can be administered to minimize or eliminate injury to an organ. In another example, SnCl₄ and iron sucrose can be administered to minimize or eliminate injury to an organ. In particular embodiments, the tin salts can be joined to the source of iron via a linker. In particular embodiments, the tin salts can be complexed with the source of iron. Also, cobalt salts can be administered in conjunction with iron sucrose to minimize or eliminate injury to an organ. To illustrate, CoCl₂ can be administered with iron sucrose to minimize or eliminate injury to an organ. In another illustrative example, CoBr₂ can be administered with iron sucrose to minimize or eliminate injury to an organ. In an additional illustrative example, CoF₂ can be administered with iron sucrose to minimize or eliminate injury to an organ. In a further example, Col₂ can be administered with iron sucrose to minimize or eliminate injury to an organ. In particular embodiments, cobalt salts can be joined to the source of iron via a linker. In some embodiments, the cobalt salts can be complexed with the iron sucrose. In particular embodiments, the tin salts, the cobalt salts, or both can be administered in conjunction with a heme protein degradation inhibitor.

In particular embodiments, the formulations, kits, and methods described herein can include conjugates, or salts thereof, that are heme protein degradation inhibitors. In particular embodiments, conjugates, or salts thereof, that function as heme protein degradation inhibitors can have the following structure:

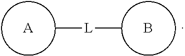

In particular embodiments, A can include a metal-carbohydrate complex. For example, A can include a saccharide complexed with the metal iron to form a metal-saccharide complex. A saccharide-metal complex can include iron in the (II) or (III) oxidation state complexed with anions of the saccharide. The anions of the saccharide can be provided from hydroxyl groups of the saccharide. In particular embodiments, one or more saccharide molecules can complex with one or more iron atoms. In cases where a compound is present in a formulation including water, iron atoms can also complex with hydroxyl groups derived from the water. The saccharide included in the metal-saccharide complex can include a monosaccharide. In other cases, the saccharide included in the metal-saccharide complex can include a disaccharide. The saccharide included in the metal-saccharide complex can include one or more saccharide units having five carbon atoms. To illustrate, the saccharide included in the metal-saccharide complex can include one or more pentose sugars. Also, the saccharide included in the metal-saccharide complex can include one or more saccharide units having six carbon atoms. To illustrate, the saccharide included in the metal-saccharide complex can include one or more hexose sugars. In illustrative examples, the metal-saccharide complex can include sucrose to form an iron-sucrose complex. In other illustrative examples, the metal-saccharide complex can include maltose to form iron carboxymaltose, iron polyisomaltose (iron dextran), or iron polymaltose (iron dextrin).

In particular embodiments, an iron-sucrose complex can have the following structure:

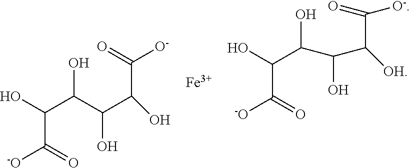

It is to be understood that although the above structure indicates a single iron atom complexed with a sucrose derived compound, one or more iron atoms can be complexed with one or more repeating units of the sucrose derived compound to balance the charges of the groups as needed.

B can include a compound having at least one nitrogen atom, at least 10 carbon atoms, and at least one oxygen atom. For example, B can include a compound having at least one nitrogen atom, at least 2 nitrogen atoms, at least 4 nitrogen atoms, at least 6 nitrogen atoms, at least 8 nitrogen atoms, at least 10 nitrogen atoms, or at least 12 nitrogen atoms. B can include a compound having no greater than 25 nitrogen atoms, no greater than 22 nitrogen atoms, no greater than 20 nitrogen atoms, no greater than 18 nitrogen atoms, or no greater than 15 nitrogen atoms. The number of nitrogen atoms included in B can include various combinations of the values provided above. In illustrative examples, B can include a compound having from 1 to 25 nitrogen atoms. In additional illustrative examples, B can include a compound having from 4 to 15 nitrogen atoms. In other illustrative examples, B can include a compound having from 4 to 10 nitrogen atoms. In further illustrative examples, B can include a compound having from 10 to 15 nitrogen atoms.

Additionally, B can include a compound having at least 10 carbon atoms, at least 15 carbon atoms, at least 18 carbon atoms, at least 20 carbon atoms, at least 22 carbon atoms, at least 25 carbon atoms, at least 28 carbon atoms, at least 30 carbon atoms, at least 32 carbon atoms, at least 35 carbon atoms, at least 38 carbon atoms, at least 40 carbon atoms, at least 42 carbon atoms, at least 45 carbon atoms, or at least 48 carbon atoms. B can also include a compound having no greater than 80 carbon atoms, no greater than 75 carbon atoms, no greater than 72 carbon atoms, no greater than 70 carbon atoms, no greater than 68 carbon atoms, no greater than 65 carbon atoms, no greater than 62 carbon atoms, no greater than 60 carbon atoms, no greater than 58 carbon atoms, no greater than 55 carbon atoms, no greater than 52 carbon atoms, or no greater than 50 carbon atoms. The number of carbon atoms included in B can include various combinations of the values provided above. In illustrative examples, B can include a compound having from 10 carbon atoms to 80 carbon atoms. In other illustrative examples, B can include a compound having from 25 carbon atoms to 50 carbons atoms. In additional illustrative examples, B can include a compound having from 30 carbon atoms to 40 carbon atoms. In further illustrative examples, B can include a compound having from 50 to 80 carbon atoms. In still further illustrative examples, B can include a compound having from 60 to 75 carbon atoms.

In particular embodiments, B can include a compound having at least one oxygen atom, at least 3 oxygen atoms, at least 5 oxygen atoms, at least 8 oxygen atoms, at least 10 oxygen atoms, or at least 12 oxygen atoms. B can also include a compound having no greater than 25 oxygen atoms, no greater than 22 oxygen atoms, no greater than 20 oxygen atoms, no greater than 18 oxygen atoms, or no greater than 15 oxygen atoms. The number of oxygen atoms included in B can include various combinations of the values provided above. In illustrative examples, B can include a compound having from 1 to 25 oxygen atoms. In other illustrative examples, B can include a compound having from 5 to 20 oxygen atoms. In additional illustrative examples, B can include a compound having from 10 to 15 oxygen atoms. In further illustrative examples, B can include a compound having from 1 to 5 carbon atoms. In still other illustrative examples, B can include a compound having from 3 to 8 oxygen atoms.

In particular illustrative examples, B can include a compound having from 2 to 5 nitrogen atoms, from 2 to 5 oxygen atoms, and from 30 to 40 carbon atoms. In other illustrative examples, B can include a compound having from 8 to 15 nitrogen atoms, from 6 to 15 oxygen atoms, and from 45 to 65 carbon atoms. B can include a compound derived from a tetrapyrrole. Additionally, B can include a metal-tetrapyrrole complex. To illustrate, B can include a complex derived from a porphyrin. In some examples, B can include a complex derived from a protoporphyrin. In other examples, B can include a complex derived from a mesoporphyrin. In still other illustrations, B can include a complex derived from a cobalamin.

L can include one or more linking components. Each linking component can bond at a site of A and a site of B. In some cases, a single linking component can bond at a single site of A and a single site of B. In other cases, multiple linking components can bond at multiple different sites of A and multiple different sites of B. For example, a first linking component can bond at a first site of A and a first site of B and a second linking component can bond at a second site of A and a second site of B. In particular embodiments, a single linking component can bond at multiple sites of A, a single linking component can bond at multiple sites of B, or a single linking component can bond at both multiple sites of A and multiple sites of B. In illustrative examples, a site of A that can bond with a linking component can include a hydroxyl site. In other illustrative examples, a site of A that can bond with a linking component can include a carbonyl site. In additional illustrative examples, a site of B that can bond with a linking component can include a hydroxyl site. In further illustrative examples, a site of B that can bond with a linking component can include a carbonyl site. In still other illustrative examples, a site of B that can bond with a linking component can include a carboxyl site. In still additional illustrative examples, a site of B that can bond with a linking component can include a phosphate site. The sites of A and the sites of B that bond with the moieties of the linking component can be neutral sites having no charge, anion sites having a negative charge, or cation sites having a positive charge. In addition, the moieties of the linking component that bond with the sites of A and the sites of B can be neutral moieties having no charge, anion moieties having a negative charge, and cation moieties having a positive charge.

Each linking component of L can include a moiety to bond at a site of A and a moiety to bond at a site of B. In particular embodiments, a moiety of a linking component that can bond at a site of A, at a site of B, or at both a site of A and a site of B can include an ester linkage. In particular embodiments, a moiety of a linking component that can bond at a site of A, a site of B, or at both a site of A and a site of B can include an amide linkage. In particular embodiments, a moiety of a linking component that can bond at a site of A, a site of B, or at both a site of A and a site of B can include a hydrazone linkage. In particular embodiments, a moiety of a linking component that can bond at a site of A, a site of B, or at both a site of A and a site of B can include an oxime linkage. A linking component of L can include a number of atoms that join a moiety bonding with a site of A and a moiety bonding with a site of B. For example, a linking component of L can include a backbone of atoms joining a moiety bonding with a site of A and a moiety bonding with a site of B. In particular embodiments, a linking component of L can include a backbone of atoms joining a moiety bonding with a site of A and a moiety bonding with a site of B including one or more carbon atoms, one or more oxygen atoms, one or more nitrogen atoms, or combinations thereof. In particular embodiments, the backbone of atoms joining a moiety bonding with a site of A and a moiety bonding with a site of B can include branches that stem from an atom included in the backbone of atoms.

L can include a linking component that forms one or more linkages with at least one site of A and at least one site of B. Examples of linkages that can be formed between L and A and L and B include ester linkages, amide linkages, hydrazone linkages, oxime linkages, or combinations thereof. Examples of a hydrazone linker used in conjunction with vitamin B12 is discussed in Bagnato J D, Eilers A L, Horton R A, Grisson C B: Synthesis and characterization of a cobalamin-colchicine conjugate as a novel tumor-targeted cytotoxid. J. Org. Chem. 2004: 69, 8987-8996.

In particular embodiments, L can include a linking component forming an ester linkage at a site of A. For example, an ester linkage between L and a site of A can have the following structure, referred to herein as Structure I:

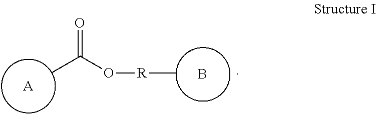

In particular embodiments, L can include a linking component forming an ester linkage at a site of B. For example, an ester linkage between L and a site of B can have the following structure, referred to herein as Structure II:

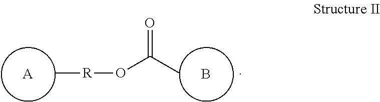

In particular embodiments, L can include a linking component forming an ester linkage at a site of A and an ester linkage at a site of B. For example, an ester linkage at a site of A and an ester linkage at a site of B can have the following structure, referred to herein as Structure III:

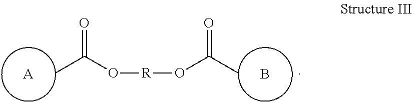

In particular embodiments, L can include a linking component forming an amide linkage at a site of A. For example, an amide linkage between L and a site of A can have the following structure, referred to herein as Structure IV:

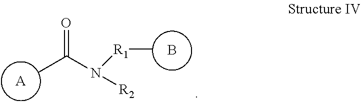

In particular embodiments, L can include a linking component having an amide linkage with a site of A. To illustrate, an amide linkage between L and a site of A can have the following structure, referred to herein as Structure V:

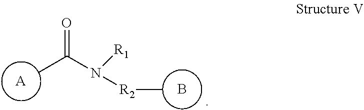

In an additional example, L can include a linking component having an amide linkage with a site of A. To illustrate, an amide linkage between L and a site of A can have the following structure, referred to herein as Structure VI:

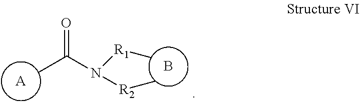

Wth regard to structure VI, a linking component of L can have a first moiety (e.g., R₁) to form a linkage with a first site of B and a second moiety (e.g., R₂) to form a linkage with a second site of B.

In particular embodiments, L can include a linking component forming an amide linkage at a site of B. For example, an amide linkage between L and a site of B can have the following structure, referred to herein as Structure VII:

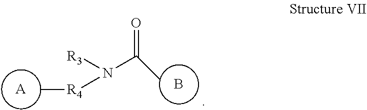

In particular embodiments, L can include a linking component forming an amide linkage at site of B. For example, an amide linkage between L and a site of B can have the following structure, referred to herein as Structure VIII:

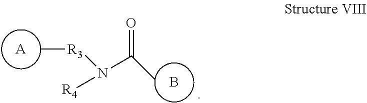

In particular embodiments, L can include a linking component having an amide linkage with a site of B. To illustrate, an amide linkage between L and a site of B can have the following structure, referred to herein as Structure IX:

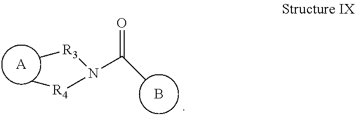

Wth regard to structure IX, a linking component of L can have a first moiety (e.g., R₃) to form a linkage with a first site of A and a second moiety (e.g., R₄) to form a linkage with a second site of A.

In particular embodiments, L can include a linking component having an amide linkage with a site of A and an amide linkage with a site of B. For example, an amide linkage between L and a site of A and an amide linkage between L and a site of B can have the following structure, referred to herein as Structure X:

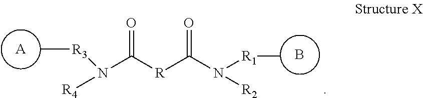

In particular embodiments, L can include a linking component having an amide linkage with a site of A and an amide linkage with a site of B. For example, an amide linkage between L and a site of A and an amide linkage between L and a site of B can have the following structure, referred to herein as Structure XI:

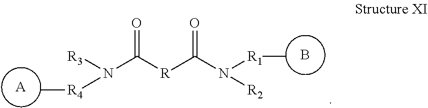

In particular embodiments, L can include a linking component having an amide linkage with a site of A and an amide linkage with a site of B. For example, an amide linkage between L and a site of A and an amide linkage between L and a site of B can have the following structure, referred to herein as Structure XII:

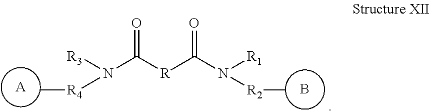

In particular embodiments, L can include a linking component having an amide linkage with a site of A and an amide linkage with a site of B. For example, an amide linkage between L and a site of A and an amide linkage between L and a site of B can have the following structure, referred to herein as Structure XIII:

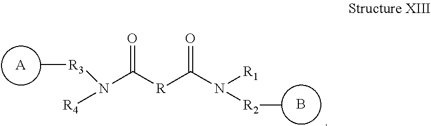

In particular embodiments, L can include a linking component having an amide linkage with a site of A and an amide linkage with a site of B. For example, an amide linkage between L and a site of A and an amide linkage between L and a site of B can have the following structure, referred to herein as Structure XIV:

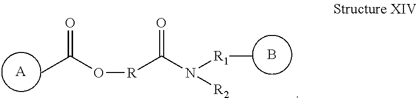

Additionally, L can include a linking component having an amide linkage with a site of A and an amide linkage with a site of B. For example, an amide linkage between L and a site of A and an amide linkage between L and a site of B can have the following structure, referred to herein as Structure XV:

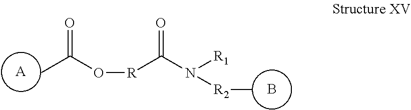

Further, L can include a linking component having an ester linkage with a site of A and an amide linkage with a first site of B and a second site of B. For example, an ester linkage between L and a site of A and an amide linkage between L and a site of B can have the following structure, referred to herein as Structure XVI:

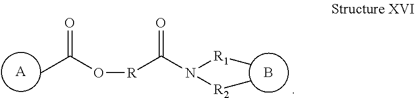

Wth regard to structure XVI, a linking component of L can have a first moiety (e.g., R₁) to form a linkage with a first site of B and a second moiety (e.g., R₂) to form a linkage with a second site of B.

In particular embodiments, L can include a linking component having an amide linkage with a site of A and an ester linkage with a site of B. For example, an amide linkage between L and a site of A and an ester linkage between L and a site of B can have the following structure, referred to herein as Structure XVII:

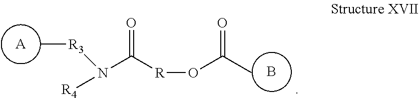

In particular embodiments, L can include a linking component having an amide linkage with a site of A and an ester linkage with a site of B. For example, an amide linkage between L and a site of A and an ester linkage between L and a site of B can have the following structure, referred to herein as Structure XVIII:

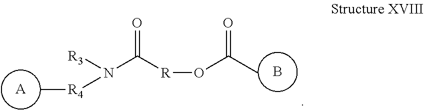

Further, L can include a linking component having an amide linkage with a first site of A and a second site of A and an ester linkage with a site of B. For example, an amide linkage between L and a site of A and an ester linkage between L and a site of B can have the following structure, referred to herein as Structure XIX:

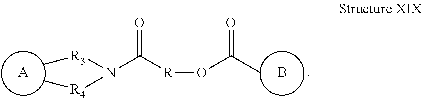

Wth regard to structure XIX, a linking component of L can have a first moiety to form a linkage with a first site of A and a second moiety to form a linkage with a second site of A.

Also, L can include a linking component having a hydrazone linkage with a site of A. For example, a hydrazone linkage between L and a site of A can have the following structure, referred to herein as Structure XX:

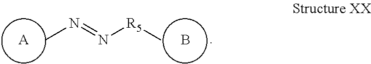

In addition, L can include a linking component having a hydrazone linkage with a site of B. For example, a hydrazone linkage between L and a site of B can have the following structure, referred to herein as Structure XXI:

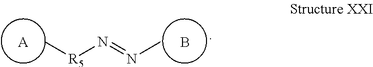

In particular embodiments, L can include a linking component having a hydrazone linkage with a site of A and a hydrazone linkage with a site of B. For example, a hydrazone linkage between L and a site of A and a hydrazone linkage between L and a site of B can have the following structure, referred to herein as Structure XXII:

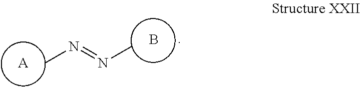

Further, L can include a linking component having an oxime linkage with a site of A. For example, an oxime linkage between L and a site of A can have the following structure, referred to herein as Structure XXIII:

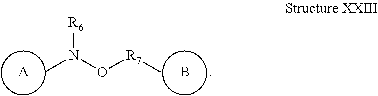

In particular embodiments, L can include a linking component having an oxime linkage with a site of B. For example, an oxime linkage between L and a site of B can have the following structure, referred to herein as Structure XXIV:

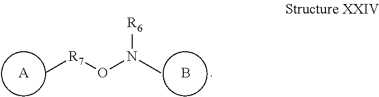

In structures I-XXIV, R, R₁, R₂, R₃, R₄, R₅, R₆, and R₇ can include at least a portion of L. In some cases, L can also include one or more atoms of the ester linkages. In particular embodiments, atoms of the ester linkages can be part of at least one of A or B.

In particular embodiments, R, R₁, R₂, R₃, R₄, R₅, R₆, R₇, or a combination thereof, can include an aliphatic chain including a number of carbon atoms. In particular embodiments, R, R₁, R₂, R₃, R₄, R₅, R₆, R₇, or a combination thereof, can include from 1 to 30 carbon atoms. In particular embodiments, R, R₁, R₂, R₃, R₄, R₅, R₆, R₇, or a combination thereof, can include from 1 to 20 carbon atoms. In particular embodiments, R, R₁, R₂, R₃, R₄, R₅, R₆, R₇, or a combination thereof, can include from 1 to 10 carbon atoms. In particular embodiments, R, R₁, R₂, R₃, R₄, R₅, R₆, R₇, or a combination thereof, can include from 1 to 8 carbon atoms. In particular embodiments, R, R₁, R₂, R₃, R₄, R₅, R₆, R₇, or a combination thereof, can include from 1 to 5 carbon atoms. The aliphatic chain can include one or more branches. In particular embodiments where the aliphatic chain includes one or more branches, each of the one or more branches can include from 1 to 10 carbon atoms. In particular embodiments where the aliphatic chain includes one or more branches, at least one branch of the one of or more branches can include a heteroatom. The heteroatom can include O, N, S, P, Cl, Br, or I. In some cases, R, R₁, R₂, R₃, R₄, R₅, R₆, R₇, or combinations thereof, can be optional.

Although various linkages are shown in Structures I-XXIV, other linkages can also be formed between L and A and L and B. Also, combinations of linkages not shown in Structures I-XXIV are also possible. For example, L can have an ester linkage with a site of A and an oxime linkage or a hydrazone linkage with a site of B. In another example, L can have an ester linkage with a site of B and an oxime linkage or a hydrazone linkage with a site of A. Additionally, L can have an amide linkage with a site of A and an oxime linkage or a hydrazone linkage with a site of B. Further, L can have an amide linkage with a site of B and an oxime linkage or a hydrazone linkage with a site of at least one of A or B.

L can include a linking component derived from a glycol. In some cases, L can include a linking component derived from a diol. In some cases, L can include a linking component derived from an ether. In particular, L can include a linking component derived from a polyether. To illustrate, L can include a linking component derived from an aliphatic polyether. In particular embodiments, L can include a linking component derived from a compound having the formula:

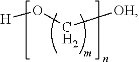

where m is from 1 to 5. In particular embodiments, when m is 1, n can be from 1 to 25. In particular embodiments, when m is 2, n can be from 1 to 15. In particular embodiments, when m is 3, n can be from 1 to 10. In particular embodiments, when m is 4, n can be from 1 to 8. In particular embodiments, when m is 5, n can be from 1 to 6. In particular embodiments, L can include a linking component derived from a polyethylene glycol having the formula:

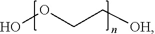

where n can from 1 to 25. In particular embodiments, n can be at least 1, at least 3, at least 5, at least 8, or at least 10. Also, n can be no greater than 25, no greater than 20, no greater than 15, or no greater than 12. In an illustrative example, n can be from 1 to 15. In another illustrative example, n can be from 1 to 10. In additional illustrative examples, n can be from 1 to 8.

L can include a linking component derived from an acid. In some cases, L can include a linking component derived from a carboxylic acid. In particular embodiments, L can include a linking component derived from formic acid, acetic acid, propionic acid, butyric acid, pentanoic acid, hexanoic acid, octanioic acid, or decanoic acid. In particular embodiments, L can include a linking component derived from a diacid. For example, L can include a linking component derived from a dicarboxylic acid. To illustrate, L can include a linking component derived from an aliphatic dicarboxylic acid. In illustrative examples, L can include a linking component derived from oxalic acid. In other illustrative examples, L can include a linking component derived from malonic acid. In additional illustrative examples, L can include a linking component derived from succinic acid. In further illustrative examples, L can include a linking component derived from glutaric acid. In still other illustrative examples, L can include a linking component derived from adipic acid. In still additional illustrative examples, L can include a linking component derived from maleic acid. In still further illustrative examples, L can include a linking component derived from fumaric acid. Also, L can include a linking component derived from phthalic acid. In some cases, L can include a linking component derived from isophthalic acid. Additionally, L can include a linking component derived from terephthalic acid.

L can include a linking component derived from one or more amino acids. For example, L can include a linking component derived from arginine, lysine, aspartic acid, glutamic acid, serine, threonine, asparagine, glutamine, or combinations thereof. In illustrative examples, L can include a linking component derived from arginine. In other illustrative examples, L can include a linking component derived from lysine. In additional illustrative examples, L can include a linking component derived from aspartic acid. In further illustrative examples, L can include a linking component derived from glutamic acid. In still other illustrative examples, L can include a linking component derived from serine. In still additional illustrative examples, L can include a linking component derived from threonine. Also, L can include a linking component derived from asparagine. In addition, L can include a linking component derived from glutamine. In an illustrative example, L can include a linking component derived from a combination of amino acids, such as a glycine-serine combination.

In particular embodiments, B can be derived from a porphyrin. For example, B can have a structure:

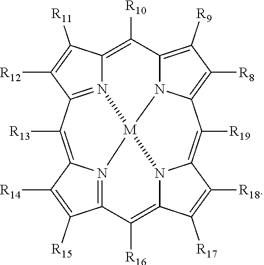

M can be a metal having a charge of ⁺2 or ⁺3. For example, M can be Sn or Zn. R₈, R₉, R₁₀, R₁₁, R₁₂, R₁₃, R₁₄, R₁₅, R₁₆, R₁₇, R₁₈, and R₁₉ can independently be a hydrogen, a halogen, a hydroxyl group, a nitro group, an amino group, an alkyl group, a cycloalkyl group, an alkenyl group, an alkoxy group, an aryl group, an aralkyl group, a carboxyl group, or a heterocyclic group. Additionally, R₈, R₉, R₁₀, R₁₁, R₁₂, R₁₃, R₁₄, R₁₅, R₁₆, R₁₇, R₁₈, and R₁₉ can be optionally substituted.

In particular embodiments, B can be linked to L through R₈, R₉, R₁₀, R₁₁, R₁₂, R₁₃, R₁₄, R₁₅, R₁₆, R₁₇, R₁₈, and R₁₉. In particular embodiments, B can be linked to L through at least one of R₈, R₉, R₁₁, R₁₂, R₁₄, R₁₅, R₁₇, or R₁₈. In particular embodiments, B can be linked to L through at least one of R₈, R₁₅, R₁₇ and R₁₈. In particular embodiments, B can be linked to L through (i) R₁₂ and R₁₄, (ii) R₁₅ and R₁₇, (iii) R₈ and R₁₈, or (iv) R₉ and R₁₁. Optionally, at least one of R₈, R₉, R₁₀, R₁₁, R₁₂, R₁₃, R₁₄, R₁₅, R₁₆, R₁₇, R₁₈, and R₁₉ can be a linking component, L.

In particular embodiments, B can include a metal protoporphyrin, a metal mesoporphyrin, a metal hematoporphyrin, a metal etioporphyrin, or a metal uroporphyrin.

“Halogen” refers to a fluorine, chlorine, bromine or iodine atom.

“Amino group” refers to an —NH₂ group. The term “amino group” also includes an amino group substituted with another atom or a group of atoms. For example, an amino group can be substituted by one or more alkyl groups.

“Alkyl group” refers to a linear or branched, saturated hydrocarbon based chain including 1 to 10 carbon atoms. The alkyl group can be a lower alkyl group including 1 to 4 carbon atoms.

“Cycloalkyl group” refers to a cyclic saturated hydrocarbon based chain including 3 to 7 carbon atoms. For example, a cycloalkly group can include a cyclohexane group or a cyclopentane group.

“Alkenyl group” refers to a linear or branched unsaturated hydrocarbon based chain including 2 to 10 carbon atoms and including one or more double bonds.

“Alkynyl group” refers to a linear or branched unsaturated hydrocarbon based chain including 2 to 10 carbon atoms and including one or more triple bonds.

“Alkoxy group” refers to an oxygen atom substituted with an alkyl group.

“Aryl group” refers to an aromatic hydrocarbon based ring or two fused aromatic hydrocarbon-based rings. The aromatic hydrocarbon based ring can include 3 to 10 carbon atoms. Examples of aryl groups include phenyl and naphthyl groups.

“Aralkyl group” refers to an alkyl substituted with an aryl group.

“Heterocyclic group” refers to a saturated or unsaturated, cyclic or polycyclic hydrocarbon based chain including one or more heteroatoms chosen from O, S, and N. The hydrocarbon based chain can include 3 to 10 carbon atoms. The term “heterocyclic group” also includes a substituted heterocyclic group.

“Heteroaryl group” refers to an aromatic heterocyclic group, such as a cyclic or polycyclic aromatic hydrocarbon based chain, including one or more heteroatoms chosen from O, S and N. Accordingly, a heteroaryl group is an example of a heterocyclic group. The aromatic hydrocarbon based chain can include 3 to 10 carbons and/or heteroatoms and one or more double bonds. The polycyclic aromatic hydrocarbon based chain includes two or more fused aromatic rings.

“Carboxyl group” refers to a group having a structure

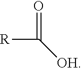

In particular embodiments, R can be a substituted or unsubstituted alkyl group.

The different “R” groups described above can be optionally substituted, for example, with a halogen, a hydroxyl group, an amino group, an amide group, a cyano group, a nitro, an alkyl group, an alkoxy group, an alkenyl group, a carboxyl group, an aryl group, a heterocyclic group, or a sulfonyl group. As an example, the “R” groups described above can be substituted with a hydroxyl group, methyl group, a carboxyl group. In particular embodiments, an alkyl group or an alkenyl group could be substituted with a carboxyl group.

Additionally, B can be derived from a mesoporphyrin. For example, B can have a structure:

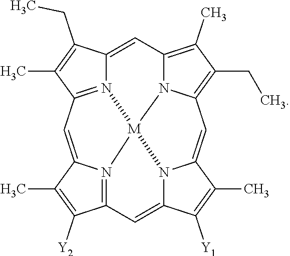

M can be a metal having a charge of ⁺2 or ⁺3. For example, M can be Sn or Zn. In particular embodiments, B can be linked to L or A through at least one of Y₁ or Y₂.

In particular embodiments, B can be derived from a protoporphyrin. In particular embodiments, B can have the following structure:

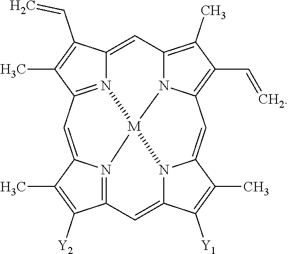

In particular embodiments, M can be a metal having a charge of ⁺2 or ⁺3. In an illustrative example, M can be Sn. In another illustrative example, M can be Zn. B can be linked to L or A through at least one of Y₁ or Y₂.

Additionally, at least one of Y₁ or Y₂ can have a structure:

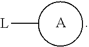

In particular embodiments, L can be a linking component having from 2 to 10 carbon atoms and A can be an iron-sucrose complex. Also, optionally, one of Y₁ or Y₂ can include H, an alkyl group having from 1 to 5 carbon atoms, or an alkyl carboxyl group having from 1 to 5 carbon atoms. In illustrative examples, at least one of Y₁ or Y₂ can have the following structure:

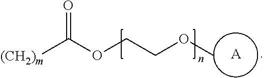

In particular embodiments, m can be from 0 to 5 and n can be from 1 to 5.

In particular embodiments, B can be derived from a protoporphyrin and be coupled to A via a linking component L having a structure:

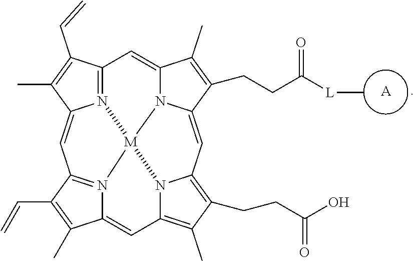

M can be a metal that includes Pt, Ni, Co, Cu, Ag, Mn, Cr, Sn, or Zn.
In particular embodiments, a linking component L can be derived from polyethylene glycol and have a structure:

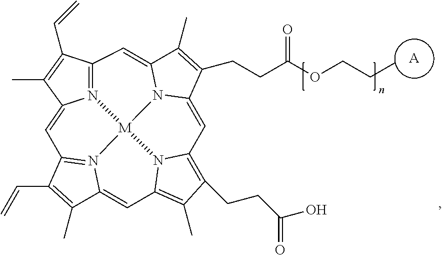

where n can be from 1 to 10.

Also, B can be derived from a protoporphyrin and be coupled to A via a linking component L having a structure:

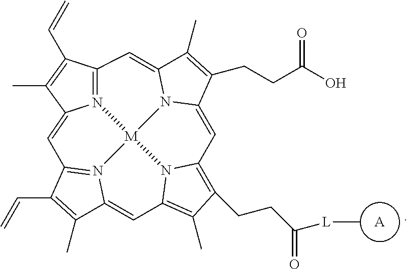

M can be a metal that includes Pt, Ni, Co, Cu, Ag, Mn, Cr, Sn, or Zn.
In particular embodiments, a linking component L can be derived from polyethylene glycol and have a structure:

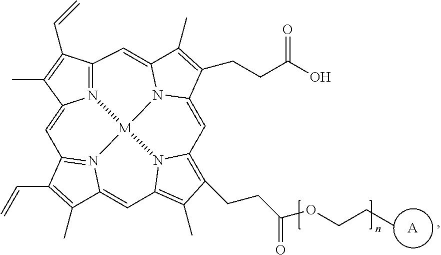

where n can be from 1 to 10.

B can be derived from a protoporphyrin and be coupled to A via a linker L having a structure:

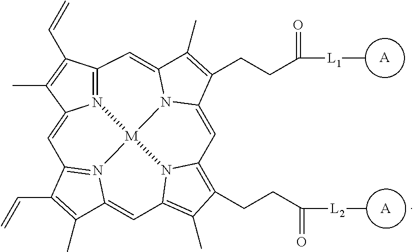

M can be a metal that includes Pt, Ni, Co, Cu, Ag, Mn, Cr, Sn, or Zn.
In particular embodiments, a linking component L can be derived from polyethylene glycol and have a structure:

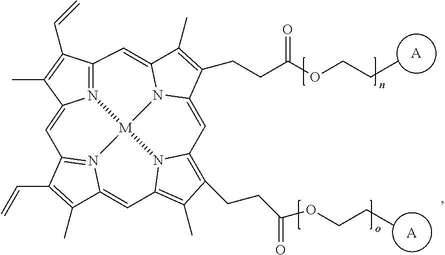

where n and o can be from 1 to 10.

In particular embodiments, B can be derived from a cobalamin and can be coupled with a saccharide-metal complex. In particular embodiments, the cobalamin is water soluble and can include a trivalent cobalt ion bound inside a corrin ring. Methylcobalamin and 5-deoxyadenosyl cobalamin are forms of cobalamins primarily used by the human body. Additional forms include adenosyl cobalamin and hydroxyl cobalamin. A cobalamin may be obtained from any appropriate synthetic or natural source, and all analogues, derivatives, salts, and prodrugs, as well as mixtures thereof. In particular embodiments, B can be derived from a cobalamin and be coupled to A via a linking component L having a structure:

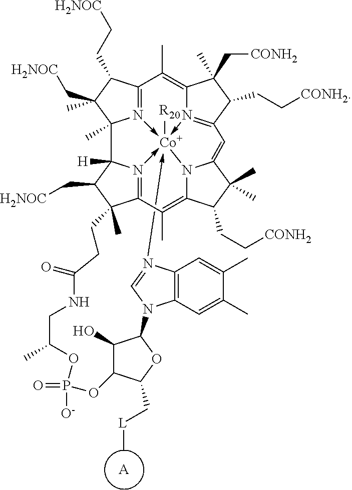

R₂₀ can include 5′-deoxyadenosyl, CH₃, OH, or CN.
In particular embodiments, a linking component L can be derived from polyethylene glycol and a structure can include:

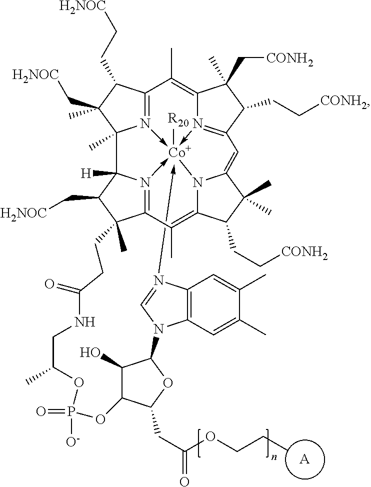

where n can be from 1 to 10.

In particular embodiments, B can be derived from a cobalamin and be coupled to A via a linking component L having a structure:

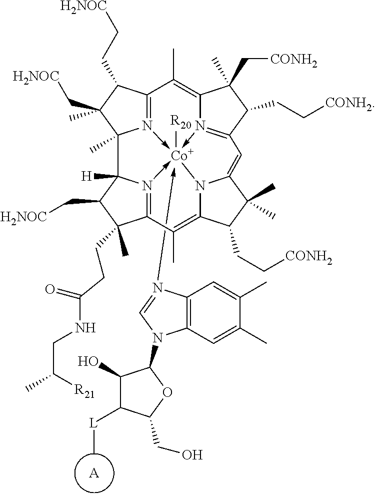

R₂₀ can include 5′-deoxyadenosyl, CH₃, OH, or CN and R₂₁ can include H, a methyl group, an ethyl group, or a hydroxyl group.
In particular embodiments, a linking component L can be derived from polyethylene glycol and a structure can include:

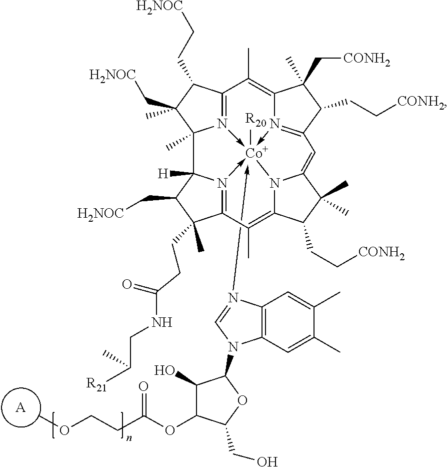

where n can be from 1 to 10.

In particular embodiments, B can be derived from a cobalamin and be coupled to A via a linking component L having a structure:

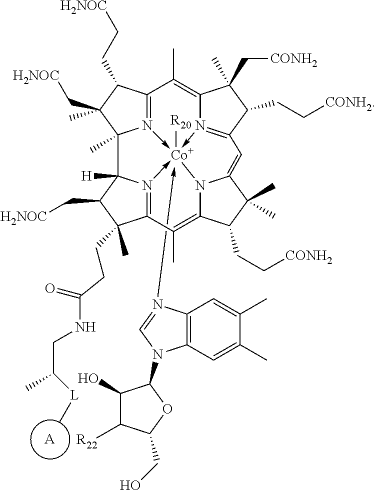

R₂₀ can be 5′-deoxyadenosyl, CH₃, OH, or CN and R₂₂ can be H, a methyl group, an ethyl group, or a hydroxyl group.
In particular embodiments, a linking component L can be derived from polyethylene glycol and a structure can include:

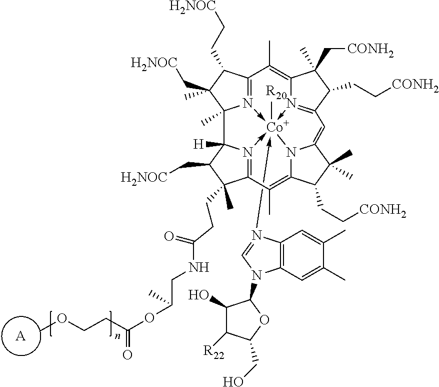

where n can be from 1 to 10.

In particular embodiments, B can be derived from a cobalamin and be coupled to A via a linker molecule L having a structure:

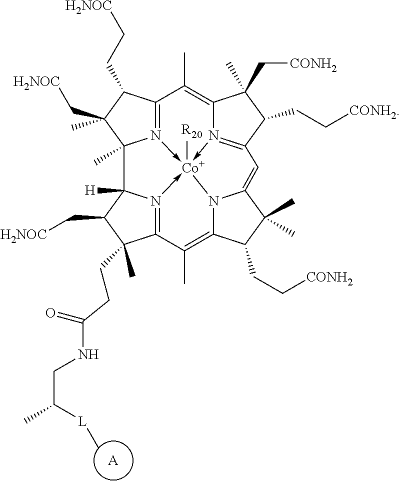

R₂₀ can include 5′-deoxyadenosyl, CH₃, OH, or CN.
In particular embodiments, a linking component L can be derived from polyethylene glycol and a structure can include:

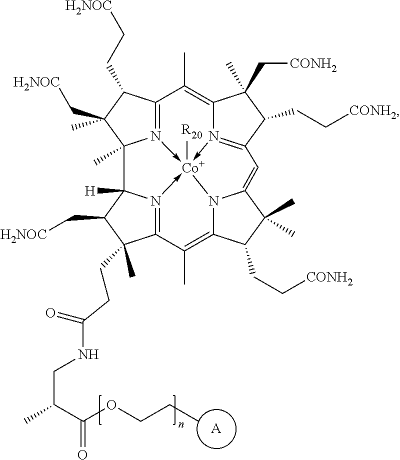

where n can be from 1 to 10.

In particular embodiments, B can be derived from a cobalamin and be coupled to A via a first linking component, L₁, and a second linking component, L₂ and have the following structure:

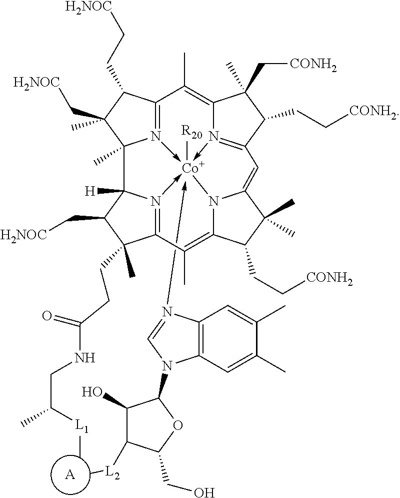

L₁ and L₂ can include any of the linking components described previously with respect to L and R₂₀ can include 5′-deoxyadenosyl, CH₃, OH, or CN.

The present disclosure describes compositions including component A and component B, wherein component A is a metal-saccharide complex, as described above. The present disclosure also describes compositions including a conjugate, wherein the conjugate includes component A and component B, wherein components A and B are linked through a linking component, L, which is as described above.

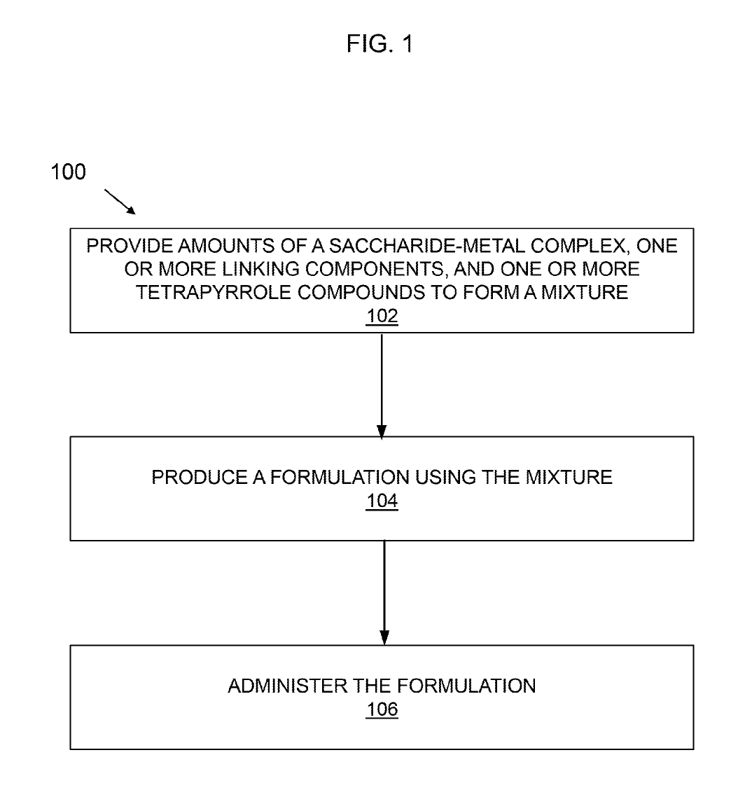

FIG. 1 includes a process 100 directed to making and using formulations including compounds linked with a saccharide-metal complex.

At 102, the process 100 includes providing amounts of a saccharide-metal complex, one or more linker molecules, and one or more tetrapyrrole compounds to form a mixture. In particular embodiments, the amounts of the saccharide-metal complex, the one or more linker molecules, and the one or more tetrapyrrole compounds can be provided such that excess saccharide-metal complex is minimized or eliminated. In particular, the amounts of a saccharide-metal complex, one or more linker molecules, and one or more tetrapyrrole compounds such that as many of the saccharide-metal complexes provided are bonded to a tetrapyrrole compound via one or more linkers. In particular embodiments, the stoichiometric ratios of the saccharide-metal complex, the one or more linker molecules, and the one or more tetrapyrrole compounds can be 1:1:1, 1.25:1:1, 1.5:1:1, 1:1.5:1, 1:1:1.5, 2:1:1, 1:2:1, 1:1:2, 5:1:1, 1:5:1, 1:1:5, 10:1:1, 1:10:1, 1:1:10, 15:1:1, 1:15:1, 1:1:15, 20:1:1, 11.25:1.25:1, 1.25:1:1.25, 1:1.25:1.25, 1.5:1.5:1, 1.5:1:1.5, 1.5:1.5:1, 2:2:1, 2:1:2, 1:2:2, 5:5:1, 5:1:5, 1:5:5, 10:10:1, 10:1:10, 15:1:15, 15:15:1, 1:15:15, 20:1:20, 20:20:1, or 1:20:20. By coupling the saccharide-metal complex with a tetrapyrrole compound via a linking component these conjugates, or salts thereof, can be delivered to an organ using a single structure rather than separately. The conjugates, or salts thereof, formed by combining the saccharide-metal complex, the one or more linking components, and the one or more tetrapyrrole compounds can function as heme protein degradation inhibitors as described previously.

In some cases, at least one of the saccharide-metal complex, the one or more linking components, or the one or more tetrapyrrole compounds can be modified before being provided. For example, enzymes can be used to cleave one or more functional groups of at least one of the saccharide-metal complex, the one or more linking components, or the one or more tetrapyrrole compounds. To illustrate, a cobalamin can be provided at 102 and a phosphatase can be provided to cleave the phosphate group of the cobalamin. In particular embodiments, one or more enzymes can be provided to cleave the benzimidazole group, the ribose group, or both from the cobalamin.

At 104, the process 100 includes producing a formulation using the mixture. In particular embodiments, producing the formulation can include producing a conjugate, or salt thereof, that includes a metal-saccharide complex coupled to a protoporphyrin-based component via one or more linking components. In various embodiments, producing the formulation can include producing a conjugate, or salt thereof, that includes a metal-saccharide complex coupled to a cobalamin-based component via one or more linking components.

Other components can be added to the mixture to produce the formulation. To illustrate, heme proteins (including modifications, variants and D-substituted analogs thereof) can be added to the mixture. Tin salts and/or cobalt salts can also be added to the mixture. The saccharide-metal complex, the one or more linking components, the one or more tetrapyrrole compounds, the heme protein, the tin salts, the cobalt salts, or combinations thereof can function as active ingredients of the formulation. In particular embodiments, the formulations include at least one heme protein and/or at least one heme protein degradation inhibitor and at least one pharmaceutically acceptable carrier. Salts and/or pro-drugs of active ingredients can also be used.

A pharmaceutically acceptable salt includes any salt that retains the activity of the active ingredient and is acceptable for pharmaceutical use. A pharmaceutically acceptable salt also refers to any salt which may form in vivo as a result of administration of an acid, another salt, or a prodrug which is converted into an acid or salt.

Suitable pharmaceutically acceptable acid addition salts can be prepared from an inorganic acid or an organic acid. Examples of inorganic acids include hydrochloric, hydroiodic, nitric, carbonic, sulfuric and phosphoric acid. Appropriate organic acids can be selected from aliphatic, cycloaliphatic, aromatic, arylaliphatic, heterocyclic, carboxylic and sulfonic classes of organic acids.

Suitable pharmaceutically acceptable base addition salts include metallic salts made from aluminum, calcium, lithium, magnesium, potassium, sodium and zinc or organic salts made from N,N′-dibenzylethylene-diamine, choline, ethylenediamine, lysine, arginine and procaine.

A prodrug includes an active ingredient which is converted to a therapeutically active compound after administration, such as by cleavage of a protein or by hydrolysis of a biologically labile group.

In particular embodiments, the formulations include active ingredients of at least 0.1% w/v or w/w of the formulation; at least 1% w/v or w/w of the formulation; at least 10% w/v or w/w of the formulation; at least 20% w/v or w/w of the formulation; at least 40% w/v or w/w of the formulation; at least 80% w/v or w/w of the formulation; at least 95% w/v or w/w of the formulation; or at least 99% w/v or w/w of the formulation.

Example generally used pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, salts, solvents or co-solvents, stabilizers, surfactants, and/or delivery vehicles.

Example antioxidants include ascorbic acid, methionine, and vitamin E.

Example buffering agents include citrate buffers, tartrate buffers, fumarate buffers, oxalate buffers, lactate buffers, phosphate buffers, histidine buffers, and/or trimethylamine salts.

An example chelating agent is EDTA.

Example isotonic agents include polyhydric sugar alcohols including trihydric or higher sugar alcohols, such as glycerin, erythritol, arabitol, xylitol, sorbitol, or mannitol.

Example preservatives include phenol, benzyl alcohol, meta-cresol, propyl paraben, octadecyldimethylbenzyl ammonium chloride, benzalkonium halides, and hexamethonium chloride.

Stabilizers refer to a broad category of excipients which can range in function from a bulking agent to an additive which solubilizes the active ingredients or helps to prevent denaturation or adherence to the container wall. Typical stabilizers can include amino acids; organic sugars or sugar alcohols; PEG; amino acid polymers; sulfur-containing reducing agents; low molecular weight polypeptides (i.e., <10 residues); proteins such as human serum albumin, bovine serum albumin, gelatin or immunoglobulins; hydrophilic polymers such as polyvinylpyrrolidone; monosaccharides; disaccharides; trisaccharides; and polysaccharides such as dextran. Stabilizers are typically present in the range of from 0.1 to 10,000 parts by weight based on active ingredient weight.

At 106, the process 100 includes administering the formulation. The formulations disclosed herein can be prepared for administration by, for example, injection, inhalation, infusion, perfusion, lavage, or ingestion. The formulations disclosed herein can further be prepared for intravenous, intradermal, intraarterial, intranodal, intralymphatic, intraperitoneal, intralesional, intraprostatic, intravaginal, intrarectal, topical, intrathecal, intratumoral, intramuscular, intravesicular, oral and/or subcutaneous administration and more particularly by intravenous, intradermal, intraarterial, intranodal, intralymphatic, intraperitoneal, intralesional, intraprostatic, intravaginal, intrarectal, intrathecal, intratumoral, intramuscular, intravesicular, and/or subcutaneous injection.

For injection, formulations can be formulated as aqueous solutions, such as in buffers including Hanks' solution, Ringer's solution, or physiological saline. The aqueous solutions can contain formulatory agents such as suspending, stabilizing, and/or dispersing agents. Alternatively, the formulations can be in lyophilized and/or powder form for constitution with a suitable vehicle, e.g., sterile pyrogen-free water, before use. Particular embodiments are prepared for intravenous administration.

For oral administration, the formulations can be prepared as tablets, pills, dragees, capsules, liquids, gels, syrups, slurries, suspensions and the like.

Formulations can be prepared as an aerosol. In particular embodiments, the aerosol is provided as part of an anhydrous, liquid or dry powder inhaler.

Formulations can also be prepared as depot preparations with suitable polymeric or hydrophobic materials (for example as an emulsion in an acceptable oil) or ion exchange resins, or as sparingly soluble derivatives, for example, as a sparingly soluble salts.

Additionally, formulations can be prepared as sustained-release systems utilizing semipermeable matrices of solid polymers containing at least one active ingredient.

Example release modifiers can include surfactants, detergents, internal phase viscosity enhancers, complexing agents, surface active molecules, co-solvents, chelators, stabilizers, derivatives of cellulose, (hydroxypropyl)methyl cellulose (HPMC), HPMC acetate, cellulose acetate, pluronics (e.g., F68/F127), polysorbates, Span® (Croda Americas, Wilmington, Del.), poly(vinyl alcohol) (PVA), Brij® (Croda Americas, Wilmington, Del.), sucrose acetate isobutyrate (SAIB), salts, and buffers.

Excipients that partition into the external phase boundary of microparticles such as surfactants including polysorbates, dioctylsulfosuccinates, poloxamers, PVA, can also alter properties including particle stability and erosion rates, hydration and channel structure, interfacial transport, and kinetics in a favorable manner.

Any formulation disclosed herein can advantageously include any other pharmaceutically acceptable carriers which include those that do not produce significantly adverse, allergic, or other untoward reactions that outweigh the benefit of administration. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety, and purity standards as required by U.S. FDA Office of Biological Standards and/or other relevant foreign regulatory agencies.

Also disclosed herein are kits including one or more containers including one or more of the active ingredients and/or formulations described herein. In particular embodiments, the kits may include one or more containers containing one or more active ingredients and/or formulations to be used in combination with the active ingredients and/or formulations described herein. Associated with such container(s) can be a notice in the form prescribed by a governmental agency regulating the manufacture, use, or sale of pharmaceuticals or biological products, which notice reflects approval by the agency of manufacture, use, or sale for human administration.

Optionally, the kits described herein further include instructions for using the kit in the methods disclosed herein. In particular embodiments, the kit may include instructions regarding preparation of the active ingredients and/or compositions for administration; administration of the active ingredients and/or compositions; appropriate reference levels to interpret results associated with using the kit; proper disposal of the related waste; and the like. In particular embodiments, possible side effects and contraindications to further use of components of the kit based on a subject's symptoms can be included. The kits and instructions can also be tailored according to the type of organ to be protected and the type of insult the organ may encounter.

In particular embodiments, the kits described herein include some or all of the necessary medical supplies needed to use the kit effectively, thereby eliminating the need to locate and gather such medical supplies. Such medical supplies can include syringes, ampules, tubing, facemask, a needleless fluid transfer device, an injection cap, sponges, sterile adhesive strips, Chloraprep, gloves, and the like. Variations in contents of any of the kits described herein can be made. Particular kits provide materials to administer formulations through intravenous administration.

As stated, the formulations, kits, and methods disclosed herein can be used to protect organs from injury by inducing acquired cytoresistance in the absence of an injury. There are numerous potential uses for the compositions, kits, and methods, some of which are described herein.

Methods disclosed herein include treating organs with active ingredients disclosed herein including salts and prodrugs thereof. Treating organs includes delivering therapeutically effective amounts. Therapeutically effective amounts include those that provide effective amounts, prophylactic treatments, and/or therapeutic treatments.

An organ is a part of a subject that is typically self-contained and has a specific vital function. Examples of organs include the heart, liver, kidneys, spleen, pancreas, brain, lungs, intestines, stomach, etc. In particular embodiments, therapeutically effective amounts can be administered directly to organs. In some cases, the organ may not be included within the subject. For example, the organ may have been removed from the subject.

Therapeutically effective amounts can also be administered to organs by administering the therapeutically effective amount to the subject in which the organ resides. Subjects include humans, veterinary animals (dogs, cats, reptiles, birds, etc.), livestock (horses, cattle, goats, pigs, chickens, etc.), and research animals (monkeys, rats, mice, fish, etc.). Treating subjects includes delivering therapeutically effective amounts. Thus, unless stated otherwise, administration to an organ can be by administration to a subject, resulting in physiological delivery to the organ or can be by administration directly to the organ.

An “effective amount” is the amount of an active ingredient or formulation necessary to result in a desired physiological change in an organ or subject. Effective amounts are often administered for research purposes. Effective amounts disclosed herein protect organs from injury by inducing acquired cytoresistance in the absence of an injury.

A “prophylactic treatment” includes a treatment administered to an organ that does not display signs or symptoms of organ injury or displays only early signs or symptoms of organ injury such that treatment is administered for the purpose of diminishing, preventing, or decreasing the risk of developing further organ injury. Thus, a prophylactic treatment functions as a preventative treatment against organ injury.

A “therapeutic treatment” includes a treatment administered to an organ that displays symptoms or signs of organ injury and is administered to the organ for the purpose of reducing the worsening of organ injury.

The actual dose amount administered to a particular organ (or subject) can be determined by a physician, veterinarian, or researcher taking into account parameters such as physical and physiological factors including target; body weight; severity of condition; upcoming insult, when known; type of organ requiring protection; previous or concurrent therapeutic interventions; idiopathy of the subject; and route of administration.

Each of the described doses of active ingredients can be a linked compound disclosed herein, a heme protein alone, heme proteins in combination, a heme protein degradation inhibitor alone, heme protein degradation inhibitors in combination or a combination of one or more heme proteins and one or more heme protein degradation inhibitors, an iron-sucrose complex, a cobalamin, a tin salt, a cobalt salt, a porphyrin, and/or Sn-PP.

In particular embodiments, organs are protected from injury during transplant. The formulations can be administered (i) to an organ donor before organ isolation from the donor; (ii) to the isolated organ before transplantation, and/or (iii) to the organ transplant recipient. This method of use can apply to any organ capable of transplant from one individual subject to a second individual subject. In particular embodiments, therapeutically effective amounts can be delivered directly to an organ following removal from a subject or prior to implantation in a second subject.

“Acute kidney injury”, (AKI) also known as “acute renal failure” (ARF) or “acute kidney failure”, refers to a disease or condition where a rapid loss of renal function occurs due to damage to the kidneys, resulting in retention of nitrogenous (urea and creatinine) and non-nitrogenous waste products that are normally excreted by the kidney. Depending on the severity and duration of the renal dysfunction, this accumulation is accompanied by metabolic disturbances, such as metabolic acidosis (acidification of the blood) and hyperkalaemia (elevated potassium levels), changes in body fluid balance, effects on many other organ systems/organ system failure, intravascular volume overload, coma and death. It can be characterized by oliguria or anuria (decrease or cessation of urine production), although nonoliguric ARF may occur. AKI is a serious complication in hospitals, resulting in a prolonged hospital stay and high mortality. Cardiac disease and cardiac surgery are both common causes of AKI. Once patients have AKI, the mortality thereof is high.

AKI may be a consequence of various causes including a) pre-renal (causes in the blood supply), which includes, hypovolemia or decreased blood volume, usually from shock or dehydration and fluid loss or excessive diuretics use; hepatorenal syndrome, in which renal perfusion is compromised due to liver failure; vascular problems, such as atheroembolic disease and renal vein thrombosis, which can occur as a complication of nephrotic syndrome; infection, usually sepsis, and systemic inflammation due to infection; severe burns; sequestration due to pericarditis and pancreatitis; and hypotension due to antihypertensives and vasodilators; b) intrinsic renal damage, which includes renal ischemia (transient blood flow reductions or interruption) toxins or medication (e.g. some NSAIDs, aminoglycoside antibiotics, iodinated contrast, lithium, phosphate nephropathy due to bowel preparation for colonoscopy with sodium phosphates); rhabdomyolysis or breakdown of muscle tissue, where the resultant release of myoglobin in the blood affects the kidney, which can also be caused by injury (especially crush injury or extensive blunt trauma), statins, stimulants and some other drugs; hemolysis or breakdown of red blood cells, which can be caused by various conditions such as sickle-cell disease, and lupus erythematosus; multiple myeloma, either due to hypercalcemia or “cast nephropathy”; acute glomerulonephritis which may be due to a variety of causes, such as anti-glomerular basement membrane disease/Goodpasture's syndrome, Wegener's granulomatosis, or acute lupus nephritis with systemic lupus erythematosus; and c) post-renal causes (obstructive causes in the urinary tract) which include, medication interfering with normal bladder emptying (e.g. anticholinergics); benign prostatic hypertrophy or prostate cancer; kidney stones; abdominal malignancy (e.g. ovarian cancer, colorectal cancer); obstructed urinary catheter; or drugs that can cause crystalluria and drugs that can lead to myoglobinuria & cystitis.

Methods of the current disclosure include protecting the kidney by inducing acquired cytoresistance. As stated, appropriate therapeutically effective amounts can initially be determined using animal models to identify dose ranges. Particular example therapeutically effective amounts of active ingredient include 10 mg/kg; 20 mg/kg; 30 mg/kg; 40 mg/kg; 50 mg/kg; 60 mg/kg; 70 mg/kg; 80 mg/kg; 90 mg/kg, and 100 mg/kg.

Example animal models of kidney injury include: glycerol-induced renal failure (mimics rhabdomyolysis); ischemia-reperfusion-induced ARF (simulates the changes induced by reduced kidney blood flow, resulting in tissue ischemia and cell tubule cell death); drug-induced models such as gentamicin, cisplatin, maleate nephrotoxicity (simulating oxidative stress+ATP deletion), NSAID, ifosfamide-induced ARF (mimics the renal failure due to clinical administration of respective drugs); uranium, potassium dichromate-induced ARF (mimics the occupational hazard); S-(1,2-dichlorovinyl)-L-cysteine-induced ARF (simulates contaminated water-induced renal dysfunction); sepsis-induced ARF (mimics the infection-induced renal failure); and radiocontrast-induced ARF (mimics renal failure in patients during use of radiocontrast media at the time of cardiac catheterization). For more information regarding these models, see Singh et al., Pharmacol. Rep. 2012, 64(1): 31-44.

Known tests of kidney function include ultrasound; and measuring lactate dehydrogenase (LDH), blood urea nitrogen (BUN), creatinine, creatinine clearance, iothalamate clearance, cystatin-C as a marker for glomerular filtration rate, and inulin clearance.

Example animal models of liver injury include: ischemic reperfusion injury; chemically-induced liver fibrosis using hepatotoxins (carbon tetrachloride, thioacetamide, dimethyl, or diethyl nitrosamine); and bile duct ligation. Further, a number of hepatotoxic compounds, including certain therapeutics, induce cytotoxicity.

In particular embodiments, the detection of biomarkers as a diagnostic of liver injury, such as injury due to ischemia, can be correlated with existing tests. These can include, for example, alkaline phosphatase (AP); 5′-nucleotidase (5′-ND); a-glutamyl transpeptidase (G-GT); leucine aminopeptidase (LAP); aspartate transaminase (AST); alanine transaminase (ALT); fructose-1,6-diphosphate aldolase (ALD); and LDH.

Additional scoring systems and parameters used to assess liver function include the Child-Pugh scoring system as follows:

Child-Pugh scoring system

| Measure | 1 point | 2 points | 3 points |
|---|---|---|---|
| Bilirubin (total) | <34 (<2) | 34-50 (2-3) | >50 (>3) |
| μmol/l (mg/dl) | | | |
| Serum albumin | >35 | 28-35 | <28 |
| g/l | | | |
| INR | <1.7 | 1.71-2.20 | >2.20 |
| Ascites | None | Mild | Severe |
| Hepatic | None | Grade I-II (or | Grade III-IV (or |
| encephalopathy | | suppressed with | refractory) |
| | | medication) | |

Example animal models of cardiac injury include: myocardial infarction (MI) models, isoproterenol cardiotoxicity, post-MI remodeling models, gene therapy models, cell therapy models, transverse aortic constriction (TAC) models, acute ischemic stroke models, renal and limb ischemia models, the Langendorff perfusion model, and the doxorubicin-induced cardiomyopathy model. See also, for example, cardiac injury animal models practiced by the Cardiovascular Research Laboratory, Baltimore, Md..

Various methods of detecting cardiac injury may be used, including non-invasive imaging, such as Magnetic Resonance Imaging (MRI), ultrasound, X-ray Computed Tomography (CT), single photon emission computed tomography (SPECT) and/or positron emission tomography (PET). Additional measures can include echocardiography, electrocardiogram, Mikro-tip pressure catheter, telemetry, immunohistochemistry, and molecular biological studies (e.g., troponin I).

Animal models of lung injury, including acute lung injury include injury inducement by intratracheal instillation of endotoxin (LPS), mechanical ventilation, hypoxemia, live bacteria (E. coli), hyperoxia, bleomycin, oleic acid, cecal ligation and puncture, and acid aspiration.

Symptoms of lung injury include labored, rapid breathing, low blood pressure, and shortness of breath. Any type of pulmonary function testing can be used. In particular embodiments, tests of pulmonary function include measuring breathing volume, arterial blood gases, and/or the A-a O₂ gradient.

Sepsis, or Systemic Inflammatory Response Syndrome (SIRS), is characterized by a whole-body inflammatory state with the presence of infection. Sepsis can lead to fever, rapid breathing and low blood pressure and can injure all organs including organs of the cardiovascular system, the immunological system and the endocrine system.

Animal models of sepsis include cecal ligation and puncture (CLP)-induced sepsis alone or in combination with instillation of bacteria (e.g., Pseudomonas aeruginosa or Streptococcus pneumoniae). Sepsis animal models also include intravenous or intraperitoneal administration of a toll-like receptor (TLR) agent such as lipopolysaccharide (LPS or endotoxin) or zymosan.

Protection against sepsis can be confirmed by measuring blood pressure, blood gasses, cytokine measurements, and secondary organ function as described elsewhere herein (e.g., lung, liver, heart, kidney function).

The Examples below are included to demonstrate particular embodiments of the disclosure. Those of ordinary skill in the art should recognize in light of the present disclosure that many changes can be made to the particular embodiments disclosed herein and still obtain a like or similar result without departing from the spirit and scope of the disclosure.

### Example Embodiments

1. A conjugate, or salt thereof, having a structure:

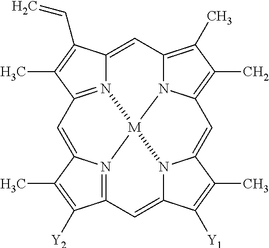

wherein:
M is selected from Sn or Zn;
at least one of Y₁ or Y₂ has a structure

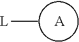

with L being a linking component having from 2 to 10 carbon atoms and A being a Fe-sucrose complex; and
optionally, one of Y₁ or Y₂ is H, an alkyl group having from 1 to 5 carbon atoms, or an alkyl carboxyl group having from 1 to 5 carbon atoms.

2. A conjugate of embodiment 1 or a salt thereof, wherein L is coupled with A via at least one ester linkage.

3. A conjugate of embodiment 1 or 2 or a salt thereof, wherein the at least one of Y₁ or Y₂ is

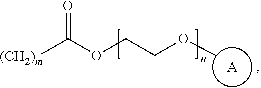

wherein m is from 0 to 5 and n is from 1 to 5.

4. A conjugate of any one of embodiments 1-3 or a salt thereof, wherein M is Sn.

5. A conjugate, or a salt thereof, having a structure:

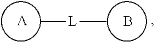

wherein A is a metal-saccharide complex, L includes one or more linking components having an aliphatic chain including less than 25 carbon atoms, and B includes a compound having at least 2 nitrogen atoms, at least 20 carbon atoms, and at least one oxygen atom.

6. A conjugate of embodiment 5 or a salt thereof, wherein B is derived from a tetrapyrrole.

7. A conjugate of embodiment 5 or 6 or a salt thereof, wherein B is derived from a protoporphyrin.

8. A conjugate of any one of embodiments 5-7 or a salt thereof, wherein B is derived from Sn-protoporphyrin.

9. A conjugate of embodiment 5 or a salt thereof, wherein B is derived from a cobalamin.

10. A conjugate of embodiment 5 or embodiment 9 or a salt thereof, wherein B is derived from a cyanocobalamin, a hydroxocobalamin, a methylcobalamin, or an adenosylcobalamin.

11. A conjugate of any one of embodiments 5-10 or a salt thereof, wherein A includes a metal complexed with a saccharide.

12. A conjugate of embodiment 11 or a salt thereof, wherein the metal is Fe.

13. A conjugate of embodiment 11 or 12 or a salt thereof, wherein the saccharide is a disaccharide.

14. A conjugate of any one of embodiments 11-13 or a salt thereof, wherein the saccharide is sucrose.

15. A conjugate of any one of embodiments 5-14 or a salt thereof, wherein B includes from 2 to 5 nitrogen atoms, from 2 to 5 oxygen atoms, and from 30 to 40 carbon atoms.

16. A conjugate of any one of embodiments 5-14 or a salt thereof, wherein B includes from 8 to 15 nitrogen atoms, from 6 to 15 oxygen atoms, and from 45 to 65 carbon atoms.

17. A c conjugate of any one of embodiments 5-16 or a salt thereof, wherein L includes a linking component that bonds at a first site of A and a second site of B.

18. A conjugate of embodiment 17 or a salt thereof, wherein the first site of A includes a hydroxyl group.

19. A conjugate of embodiment 17 or a salt thereof, wherein the first site of A includes a carbonyl group.

20. A conjugate of any one of embodiments 17-19 or a salt thereof, wherein the second site of B includes a carboxyl group.

21. A conjugate of any one of embodiments 17-19 or a salt thereof, wherein the second site of B includes a hydroxyl group.

22. A conjugate of any one of embodiments 17-19 or a salt thereof, wherein the second site of B includes a phosphate group.

23. A conjugate of any one of embodiments 5-16 or a salt thereof, wherein L includes a first linking component that bonds at a first site of A and a second site of B and a second linking component that bonds at a third site of A and a fourth site of B.

24. A conjugate of embodiment 23 or a salt thereof, wherein the first site of A includes a hydroxyl group or a carbonyl group and the third site of A include a hydroxyl group or a carbonyl group.

25. A conjugate of embodiment 23 or 24 or a salt thereof, wherein the second site of B and the fourth site of B include a carboxyl group, a hydroxyl group, a phosphate group, or a combination thereof.

26. A conjugate of any one of embodiments 5-16 or a salt thereof, wherein L includes a linking component that bonds at a first site of A, a second site of B, and a third site of B.

27. A conjugate of any one of embodiments 5-16 or a salt thereof, wherein L includes a linking component that bonds at a first site of A, a second site of A, and a third site of B.

28. A conjugate of any one of embodiments 5-16 or a salt thereof, wherein L includes a linking component that bonds at a first site of A, a second site of A, a third site of B, and a fourth site of B.

29. A conjugate of any one of embodiments 5-28 or a salt thereof, wherein A is joined to L via an ester linkage.

30. A conjugate of any one of embodiments 5-29 or a salt thereof, wherein B is joined to L via an ester linkage.

31. A conjugate of any one of embodiments 5-22 or a salt thereof, where A is joined to L via an ester linkage and B is joined to L via an ester linkage.

32. A conjugate of any one of embodiments 5-28 or 30 or a salt thereof, wherein A is joined to L via an amide linkage.

33. A conjugate of any one of embodiments 5-29 or a salt thereof, wherein B is joined to L via an amide linkage.

34. A conjugate of any one of embodiments 5-28 or a salt thereof, wherein A is joined to L via an amide linkage and B is joined to L via an amide linkage.

35. A conjugate of any one of embodiments 5-22, 30, or 33 or a salt thereof, wherein A is joined to L via a hydrazone linkage.

36. A conjugate of any one of embodiments 5-22 or 32 or a salt thereof, wherein B is joined to L via a hydrazone linkage.

37. A conjugate of any one of embodiments 5-22 or a salt thereof, wherein A is joined to L via a hydrazone linkage and B is joined to L via a hydrazone linkage.

38. A conjugate of any one of embodiments 5-22, 30, 33, or 36 or a salt thereof, wherein A is joined to L via an oxime linkage.

39. A conjugate of any one of embodiments 5-22, 32, or 35 or a salt thereof, wherein, B is joined to L via an oxime linkage.

40. A conjugate of any one of embodiments 5-22 or a salt thereof, wherein A is joined to L via an oxime linkage and B is joined to L via an oxime linkage.

41. A conjugate of any one of embodiments 5-40 or a salt thereof, wherein L includes an aliphatic chain having from 1 to 20 carbon atoms.

42. A conjugate of any one of embodiments 5-41 or a salt thereof, wherein L is derived from a diol.

43. A conjugate of any one of embodiments 5-42 or a salt thereof, wherein L is derived from an aliphatic diol having a structure

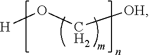

wherein m is from 1 to 5 and n is from 1 to 25.

44. A conjugate of any one of embodiments 5-43 or a salt thereof, wherein L is derived from a polyethylene glycol having a structure

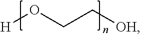

wherein n is from 1 to 25.

45. A conjugate of any one of embodiments 5-41 or a salt thereof, wherein L is derived from a diacid.

46. A conjugate of any one of embodiments 5-41 or 45 or a salt thereof, wherein L is derived from an aliphatic dicarboxylic acid.

47. A conjugate of any one of embodiments 5-41, 45, or 46 or a salt thereof, wherein L is derived from oxalic acid, malonic acid, succinic acid, glutaric acid, adipic acid, maleic acid, fumaric acid, phthalic acid, isophthalic acid, terephthalic acid, or combinations thereof.

48. A conjugate of any one of embodiments 5-41 or a salt thereof, wherein L is derived from an amino acid.

49. A conjugate of any one of embodiments 5-41 and 48 or a salt thereof, wherein L is derived from arginine, lysine, aspartic acid, glutamic acid, serine, threonine, asparagine, glutamine, or combinations thereof.

50. A conjugate, or a salt thereof, having a structure

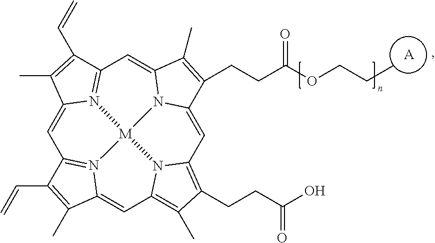

wherein n is 1 to 5 and A is an Fe-sucrose complex.

51. A conjugate, or a salt thereof, having a structure

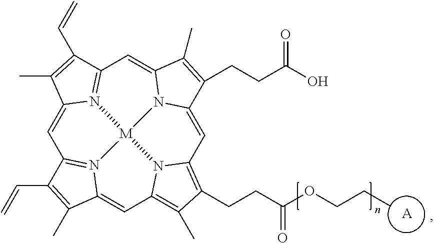

wherein n is 1 to 5 and A is a Fe-sucrose complex.

52. A conjugate, or a salt thereof, having a structure

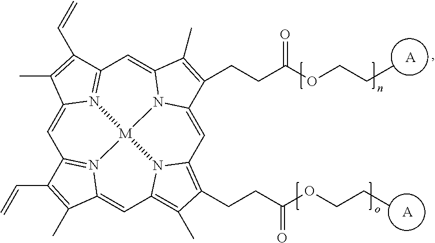

wherein n is 1 to 5, o is 1 to 5, and A is a Fe-sucrose complex.

53. A conjugate, or a salt thereof, having a structure

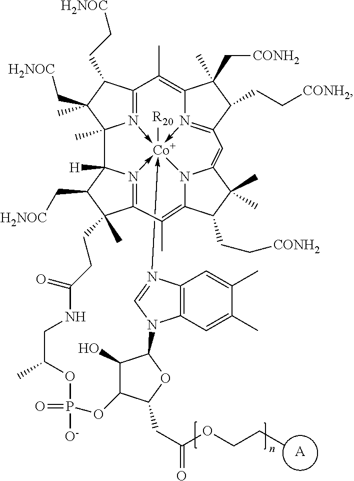

wherein R₂₀ includes 5′-deoxyadenosyl, CH₃, OH, or CN, n is from 1 to 10, and A is a Fe-sucrose complex.

54. A conjugate, or a salt thereof, having a structure

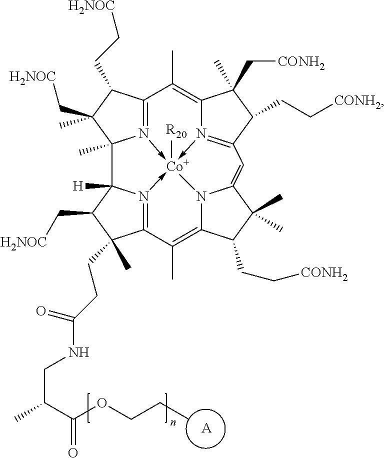

wherein R₂₀ includes 5′-deoxyadenosyl, CH₃, OH, or CN, n is from 1 to 10, and A is a Fe-sucrose complex.

55. A conjugate, or a salt thereof, having a structure

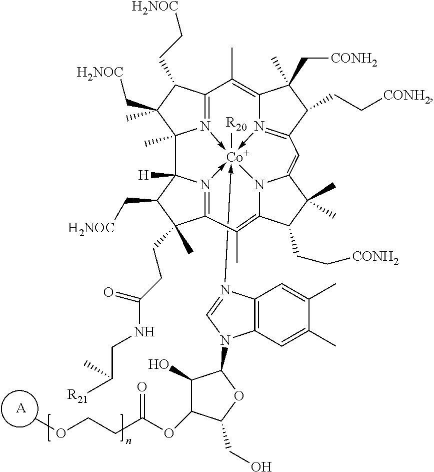

wherein R₂₀ includes 5′-deoxyadenosyl, CH₃, OH, or CN, R₂₁ includes H, a methyl group, an ethyl group, or a hydroxyl group, n is from 1 to 10, and A is a Fe-sucrose complex.

56. A conjugate, or a salt thereof, having a structure

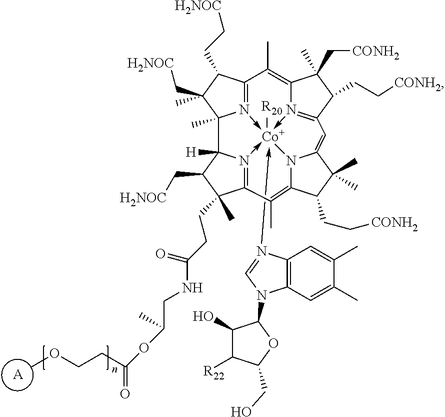

wherein R₂₀ includes 5′-deoxyadenosyl, CH₃, OH, or CN, R₂₂ includes H, a methyl group, an ethyl group, or a hydroxyl group, n is from 1 to 10, and A is a Fe-sucrose complex.

57. A kit including a therapeutically effective amount of a formulation including a conjugate or a salt thereof t of any one of embodiments 1 to 56, wherein administration of the therapeutically effective amount of the compound to an organ protects the organ without causing injury to the organ.

58. A kit of embodiment 57, wherein administration of the therapeutically effective amount of the formulation generates acquired cytoresistance in the organ in the absence of causing injury to the organ

59. A kit of embodiment 57 or 58, wherein administration of the therapeutically effective amount of the formulation to the organ up-regulates expression of protective stress proteins in the organ without causing injury to the organ.

60. A kit of any one of embodiments 57-59, further including administration instructions.

61. A method including:
administering to an organ, a therapeutically effective amount of a formulation including a conjugate of any one of embodiments 1-56 or a salt thereof, wherein administering the therapeutically effective amount of the formulation to the organ protects the organ from the injury without causing injury to the organ.

62. A method of embodiment 61, wherein administering the therapeutically effective amount of the formulation to the organ generates acquired cytoresistance in the organ without causing injury to the organ.

63. A method of embodiment 61 or 62, wherein administering the therapeutically effective amount of the formulation to the organ up-regulates expression of protective stress proteins in the organ without causing injury to the organ.

64. A method of any one of embodiments 61-63, wherein the formulation includes a heme protein.

65. A method of any one of embodiments 61-63, wherein the injury is an injury based on an insult.

66. A method of embodiment 65, wherein the insult is scheduled.

67. A method of embodiment 65, wherein administering the therapeutically effective amount of the formulation to the organ occurs at least 8 hours before the scheduled insult.

68. A method of embodiment 65, wherein the scheduled insult is surgery, chemotherapy, or radiocontrast toxicity.

69. A method of embodiment 68, wherein the surgery is an organ transplant surgery.

70. A method of any one of embodiments 61-63, wherein the organ is a heart, kidney, liver, or lung.

71. A method of any one of embodiments 61-63, wherein the organ is a kidney and protection is evidenced by prevention or reduction in BUN or serum creatinine increases as compared to a reference level.

72. A method of embodiment 61, wherein the formulation includes a modified heme protein.

73. A method of embodiment 72, wherein the modified heme protein is a nitrited heme protein or a PEGylated heme protein.

74. A method of embodiment 64, wherein the heme protein is myoglobin.

75. A method of embodiment 64, wherein the heme protein is a myoglobin variant or a myoglobin modification.

76. A method of embodiment 75, wherein the myoglobin modification is a nitrited myoglobin or a PEGylated myoglobin

77. A method includes:
providing amounts of a saccharide-metal complex, one or more linking components, and one or more tetrapyrrole compounds to form a mixture;
producing a formulation including a conjugate of any one of embodiments 1-56 or a salt thereof using the mixture;
administering the formulation.

78. A method of embodiment 77, wherein amounts of the saccharide-metal complex, the one or more linking components, and the one or more tetrapyrrole compounds are provided in a 1:1:15 ratio.

79. A method of embodiment 77 or 78, wherein administering the formulation includes cleaving a first linkage between A and L, a second linkage between B and L, or cleaving both the first and second linkage.

80. A method of any one of embodiments 77-79, wherein the one or more tetrapyrrole compounds include a cobalamin and providing an amount of the cobalamin includes contacting the cobalamin with a phosphatase.

81. A method of any one of embodiments 77-80, wherein the one or more tetrapyrrole compounds include a cobalamin and providing an amount of the cobalamin includes providing one or more enzymes to cleave the benzimidazole group and the ribose group from the cobalamin.

82. A kit including a therapeutically effective amount of a formulation including (i) a source of iron and (ii) a tin salt or a cobalt salt, wherein administration of the therapeutically effective amount of the formulation to an organ protects the organ without causing injury to the organ.

83. A kit of embodiment 82, wherein administration of the therapeutically effective amount of the formulation generates acquired cytoresistance in the organ in the absence of causing injury to the organ

84. A kit of embodiment 82 or 83, wherein administration of the therapeutically effective amount of the formulation to the organ up-regulates expression of protective stress proteins in the organ without causing injury to the organ.

85. A kit of any one of embodiments 82-84, further including administration instructions.

86. A kit of any one of embodiments 82-85, wherein the source of iron includes iron sucrose.

87. A kit of any one of embodiments 82-86, wherein the tin salt includes SnCl₂ or SnCl₄.

88. A kit of any one of embodiments 82-86, wherein the cobalt salt includes CoCl₂, CoBr₂, CoF₂, or Col₂.

89. A kit of any one of embodiments 82-88, wherein the formulation further includes a protoporphyrin.

90. A kit of embodiment 89, wherein the protoporphyrin includes Sn-protoporphyrin.

91. A kit of any one of emdodiments 82-90, wherein the formulation further includes a cobalamin or a modified cobalamin.

92. A kit of any one of embodiments 82-91, wherein the formulation further includes a composition of any one of embodiments 1-56.

93. A method including:
administering to an organ, a therapeutically effective amount of a formulation including (i) a source of iron and (ii) a tin salt or a cobalt salt, wherein administration of the therapeutically effective amount of the formulation to the organ protects the organ without causing injury to the organ.

94. A method of embodiment 93, wherein administration of the therapeutically effective amount of the formulation generates acquired cytoresistance in the organ in the absence of causing injury to the organ

95. A method of embodiment 93 or 94, wherein administration of the therapeutically effective amount of the formulation to the organ up-regulates expression of protective stress proteins in the organ without causing injury to the organ.

96. A method of any one of embodiments 93-95, wherein the organ is protected from an injury based on an insult.

97. A method of embodiment 96, wherein the insult is scheduled.

98. A method of embodiment 97, wherein the administration occurs at least 8 hours before the scheduled insult.

99. A method of embodiment 97, wherein the scheduled insult is surgery, chemotherapy, or radiocontrast toxicity.

100. A method of embodiment 99, wherein the surgery is an organ transplant surgery.

101. A method of any one of embodiments 93-95, wherein the organ is a transplanted organ.

102. A method of any one of embodiments 93-95, wherein the organ is a heart, kidney, liver, or lung.

103. A method of any one of embodiments 93-95, wherein the organ is a kidney and protection is evidenced by prevention or reduction in blood urea nitrogen (BUN) or serum creatinine increases as compared to a reference level.

104. A method of any one of embodiments 93-95, wherein the formulation further includes a heme protein.

105. A method of embodiment 104, wherein the heme protein is a heme protein variant, a heme protein d-substituted analog, a heme protein modification, or combination thereof.

106. A method of embodiment 104, wherein the heme protein is a modified heme protein.

107. A method of embodiment 106, wherein the modified heme protein is a nitrited heme protein or a PEGylated heme protein.

108. A method of embodiment 104, wherein the heme protein is myoglobin.

109. A method of embodiment 104, wherein the heme protein is a myoglobin variant or a myoglobin modification.

110. A method of any one of embodiments 93-109, wherein the source of iron includes iron sucrose.

111. A method of any one of embodiments 93-110, wherein the tin salt includes SnCl₂ or SnCl₄.

112. A method of any one of embodiments 93-111, wherein the cobalt salt includes CoCl₂, CoBr₂, CoF₂, or Col₂.

113. A method of any one of embodiments 93-112, wherein the formulation further includes a protoporphyrin.

114. A method of embodiment 113, wherein the protoporphyrin includes Sn-protoporphyrin.

115. A method of any one of embodiments 93-114, wherein the formulation further includes a cobalamin or a modified cobalamin.

116. A method of any one of embodiments 93-115, wherein the formulation further includes a conjugate, or a salt thereof, of any one of embodiments 1-56.

117. A conjugate, or a salt thereof, including component A, component B, and a linking component L, wherein component A and component B are linked through L, and wherein:
component A includes a metal-saccharide complex;
linking component L includes an aliphatic chain of 1 to 25 carbon atoms;
component B is a compound having a formula

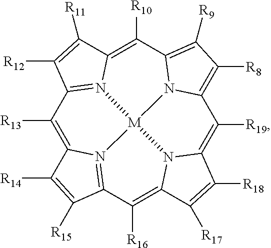

wherein:
M is a metal ion having a charge of ⁺2 or ⁺3;
R₈, R₉, R₁₀, R₁₁, R₁₂, R₁₃, R₁₄, R₁₅, R₁₆, R₁₇, R₁₈, or R₁₉ is independently a hydrogen, a halogen, a hydroxyl group, a nitro group, an amino group, an alkyl group, a cycloalkyl group, an alkenyl group, an alkynyl group, an alkoxy group, an aryl group, an aralkyl group, or a heterocyclic group, and R₈, R₉, R₁₀, R₁₁, R₁₂, R₁₃, R₁₄, R₁₅, R₁₆, R₁₇, R₁₈, or R₁₉ is optionally substituted; and
B is linked to L through R₈, R₉, R₁₁, R₁₂, R₁₄, R₁₅, R₁₇, or R₁₈.

118. A conjugate of embodiment 117 or a salt thereof, wherein the metal of component A is iron.

119. A conjugate of embodiment 117 or a salt thereof, wherein component A is an iron-sucrose complex.

120. A conjugate of any of embodiments 117-119 or a salt thereof, wherein component B is a metal protoporphyrin, metal mesoporphryin, metal hematoporphyrin, metal etioporphyrin, or metal uroporphyrin.

121. A conjugate of embodiment 120 or a salt thereof, wherein component B is a metal protoporphryin or a metal mesoporphyrin.

122. A conjugate of embodiment 120 or 121 or a salt thereof, wherein the metal of component B is Sn or Zn.

123. A conjugate of any of embodiments 117-122, wherein L is derived from an aliphatic diol having the formula

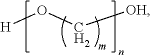

wherein m is an integer from 1 to 5 and n is an integer from 1 to 25.

124. A conjugate of any of embodiments 117-122, wherein L is derived from a polyethylene glycol having the formula

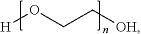

wherein n is an integer from 1 to 25.

125. A composition including the conjugate of any of embodiments 117-124 or a salt thereof and a carrier.

126. A pharmaceutical composition including the conjugate of any of embodiments 117-124 or a salt thereof and a pharmaceutically acceptable carrier.

127. A pharmaceutical composition of embodiment 126, wherein the pharmaceutical composition further includes a heme protein.

128. A pharmaceutical composition of embodiment 127, wherein the heme protein is hemoglobin or myoglobin.

129. A kit including the pharmaceutical composition of any of embodiments 126-128 and a container and/or instructions for using the kit.

130. A kit of embodiment 129, wherein the kit further includes a heme protein.

131. A kit of embodiment 130, wherein the hemeprotein is a hemoglobin or a myoglobin.

132. A method of protecting an organ from injury, the method including administering to the organ a therapeutically effective amount of a formulation including the conjugate of any of embodiments 117-124 or a salt thereof prior to occurrence of the injury, wherein the administering protects the organ from the injury without causing injury to the organ.

133. A method of inducing ischemic preconditioning in a subject, the method including administering to the subject in need thereof a therapeutically effective amount of a formulation including the conjugate of any of embodiments 117-124 or a salt thereof prior to occurrence of an insult, wherein the administering induces ischemic preconditioning in the subject without causing injury to the subject.

134. A method of inducing acquired cytoresistance in a subject, the method including administering to the subject in need thereof a therapeutically effective amount of a formulation including the conjugate of any of embodiments 117-124 or a salt thereof prior to occurrence of an insult, wherein the administering induces acquired cytoresistance in the subject without causing injury to the subject.

135. A method of embodiment 134, wherein the insult is surgery, chemotherapy, or radiocontrast toxicity.

136. A method of embodiment 135, wherein the surgery is transplantation of an organ.

137. A method of embodiment 136, wherein the organ is kidney, heart, liver, or lung.

138. A method of embodiment 134, wherein the insult is sepsis.

### Experimental Examples

### Example 1

Effects of Fe sucrose and cyanocobalamin (Vitamin B₁₂) on heme oxygenase-1 induction in kidney. Heme oxygenase-1 (HO-1) upregulation is a critical mediator of N-myoglobin/SnPP's cytoprotective activity in kidney and extra-renal organs. Hence, additional agents were sought that can induce HO-1 up-regulation, and thus, contribute to the emergence of tissue protection against toxic and ischemic forms of injury. Because Fe is the critical mediator of N-Mgb's activity, the impact of an Fe-carbohydrate polymer (Fe sucrose; molecular weight ranging from 34-61 kDa) on HO-1 levels was assessed.

As an alternative and/or complementary strategy, the impact of cyanocobalamin (vitamin B12) on HO-1 induction in kidney was studied. The rationale for B12 testing is that both cobalt and cyanide can independently induce HO-1. Thus, B12 could represent a safe method to administer both cyanide and cobalt, and as a single agent, since both are integral parts of the B12 molecule.

Methods. Male CD-1 mice (25-40 grams) Charles River, Wilmington, Mass.) were used for all experiments. They were housed under standard vivarium conditions with free food and water access. All experiments were approved by the Fred Hutchinson Cancer Research Center IACUC in accordance with NIH guidelines.

Effects of Fe sucrose (FeS)/tin protoporphyrin (SnPP) on AKI severity. Maleate model of AKI. When injected into rodents, maleate undergoes relatively selective proximal tubule cell uptake via organic anion transporters. Once intracellular accumulation occurs, maleate is a preferred substrate for succinyl-CoA:3-oxoacid CoA transferase. This results in the formation of maleyl-coenzyme A. With subsequent conversion of maleyl CoA into a stable thioether, severe coenzyme A (CoA) depletion results. Ample levels of CoA are essential for fatty acid “activation”, allowing for their subsequent metabolism through the Krebs cycle, yielding ATP. In the absence of this process, proximal tubule ATP depletion and cell injury result. Additionally, maleate conjugates the sulfhydryl group of glutathione (GSH), culminating in GSH depletion and potential oxidant tubular stress.

The following experiment tested whether FeS, SnPP or combined FeS+SnPP can mitigate this form of acute kidney injury (AKI). Twenty seven mice were subjected to 200 μL IV tail injections of one of the following: 1) vehicle (phosphate buffered saline, PBS; n, 10); 2) 1 mg FeS (American Regent (Shirley, N.Y.; n, 3); 3) 1 μmole SnPP (Frontier Scientific, Logan, Utah; n 7), or FeS+SnPP, n, 7). Eighteen hrs later, all mice received an IP injection of Na maleate (800 mg/Kg; in 500 ul of PBS). Eighteen hrs later, the mice were deeply anesthetized with pentobarbital (50 mg/Kg IP), the abdominal cavities were opened, and blood samples were obtained from the abdominal vena cava. The severity of kidney injury was assessed by determining plasma blood urea nitrogen (BUN) and plasma creatinine (PCr) concentrations.

Renal ischemic—reperfusion injury (IRI) model of AKI. The following experiment assessed whether combination FeS+SnPP can mitigate the renal artery occlusion model of AKI. Mice received 200 μl tail vein injections of either PBS (n, 9) or FeS+SnPP (n, 8), as noted above. Eighteen hrs later, the mice were deeply anesthetized with pentobarbital (40-50 mg/Kg IP), the abdominal cavities were opened, the renal pedicles were identified and both were occluded with microvascular clamps. Body temperature was maintained at 36-37° C. throughout. Following 22 minutes of bilateral renal ischemia, the clamps were removed, uniform reperfusion was visually confirmed by the reappearance of a normal renal color (loss of tissue cyanosis), and then the abdominal cavities were closed in two layers with silk suture. Eighteen hrs later, the mice were re-anesthetized, the abdominal cavities were re-opened, and terminal blood samples were obtained from the vena cava. The severity of renal injury was determined by BUN and PCr concentrations.

FeS/B12 effects on the severity of AKI. Glycerol model of AKI. Mice received tail vein injections of either PBS vehicle (n 6), or combination FeS+1 μmole B12 (n, 6; B12 from Alfa Aesar, Ward Hill, Mass.). Eighteen hrs later, the mice were lightly anesthetized with isoflurane, and then the glycerol model of rhabdomyolysis AKI was induced (9 ml/Kg 50% glycerol, administered in two equally divided IM injections into the upper hind limbs). Eighteen hrs post glycerol injection, the mice were deeply anesthetized with pentobarbital and terminal vena cava blood samples were obtained. Renal injury severity was gauged by terminal BUN and PCr concentrations.

Maleate model of AKI. Mice received tail vein injections of either combination of FeS+B12 or vehicle (n, 6 per group). Eighteen hrs later they received IP maleate injections, as noted above. The severity of AKI was determined 18 hrs post maleate injection by terminal BUN and PCr assessments.

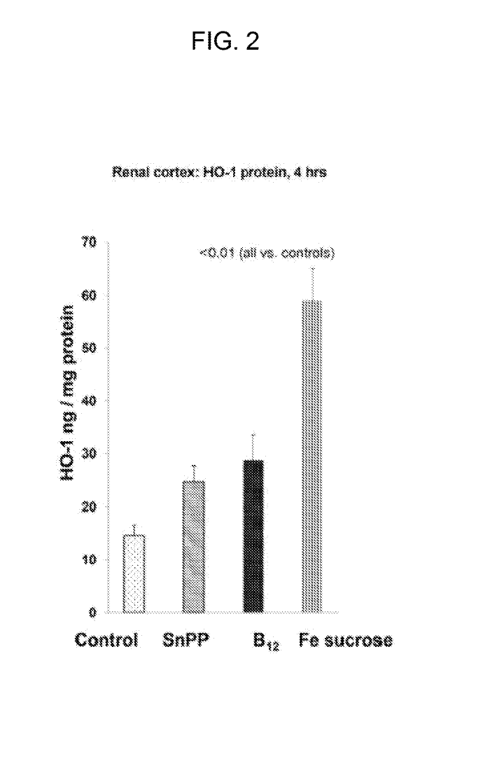

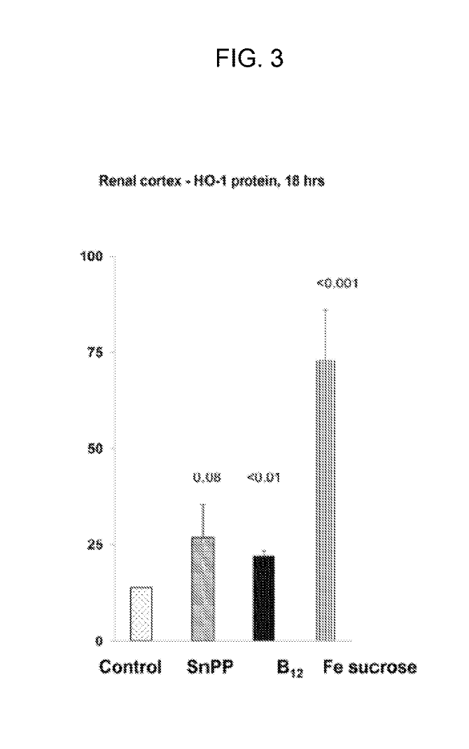

Effects of FeS, SnPP, and B12 on renal cortical induction of heme oxygenase 1 (HO-1). The following experiments assessed the effects of FeS, SnPP, and B12 on the possible induction of the cytoprotective protein HO-1. To this end, mice were injected with each of these agents, as noted above. Either 4 or 18 hrs later, they were anesthetized and the kidneys were removed through a midline abdominal incision. The renal cortices were dissected on ice and then extracted for protein and mRNA. The samples were then assayed for HO-1 protein by ELISA, and HO-1 mRNA by RT-PCR, factored by GAPDH levels. Five normal mice provided control values. The results are shown in FIG. 2 and FIG. 3.

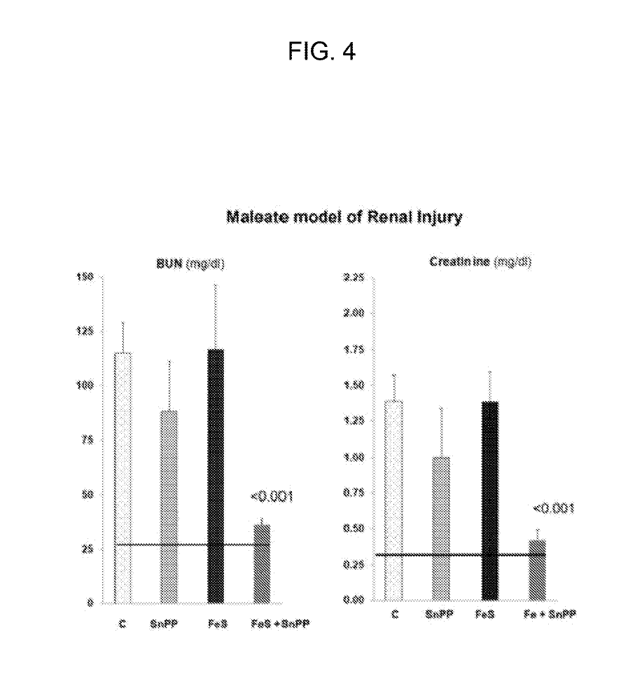

Effects of FeS/SnPP on the severity of AKI. Maleate-induced AKI: As shown in FIG. 4, maleate injection caused severe AKI as denoted by marked BUN and PCr increases over maleate injected controls (C). Neither SnPP alone nor FeS alone significantly altered the severity of renal injury. However, combined FeS+SnPP conferred marked protection, as denoted by 75% reductions in BUN/PCr concentrations (the horizontal lines represent the means of BUN/PCr levels in normal mice).

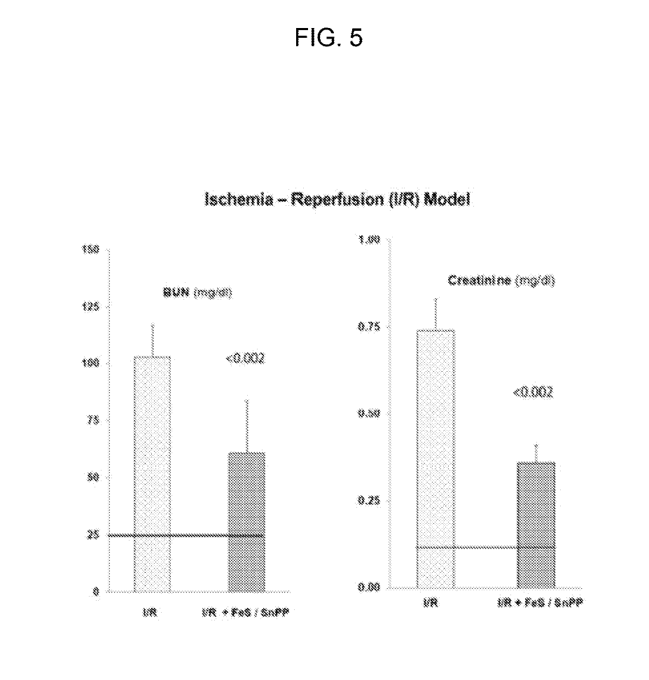

Renal ischemia-reperfusion (IRI) induced AKI: Within 18 hrs of inducing IRI, 4 fold elevations in BUN and PCr concentrations resulted (FIG. 5). Pre-treatment with FeS+SNPP conferred significant protection, lowering the BUN and PCr levels by 50%. The horizontal lines represent mean BUN/PCr levels in normal mice.

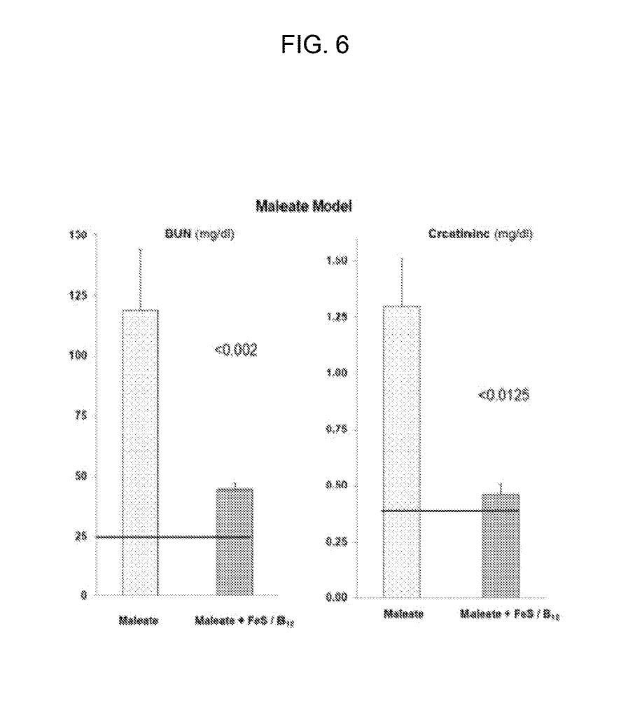

Effects of FeS/B12 on AKI severity. Maleate induced AKI: Again, maleate injection induced severe AKI (FIG. 6). Pre-treatment with FeS+B12 markedly mitigated this injury, as denoted by BUN/PCr reductions. The horizontal lines represent mean BUN/PCr levels in normal mice.

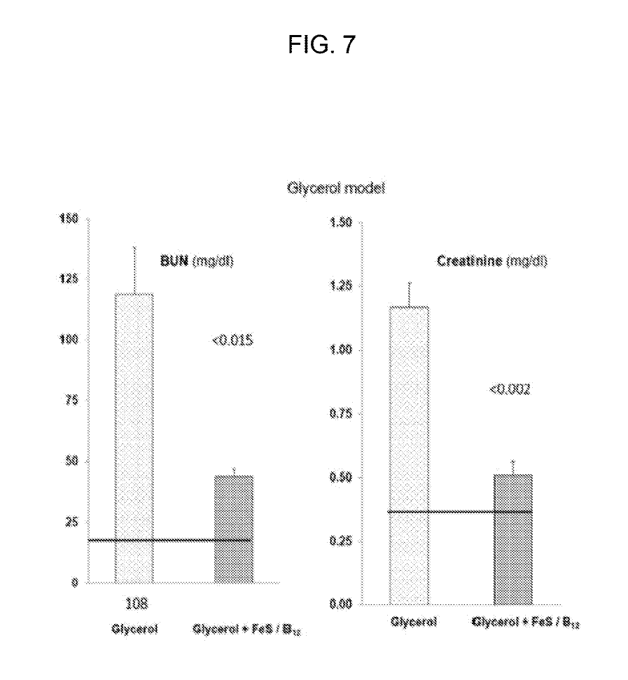

Glycerol model of AKI: Severe renal failure resulted within 18 hrs of glycerol injection (FIG. 7). Pre-with FeS+B12 conferred substantial functional protection, as denoted by marked reductions in both 18 hr BUN and PCr concentrations. The horizontal lines represent mean BUN/PCr levels for normal mice.

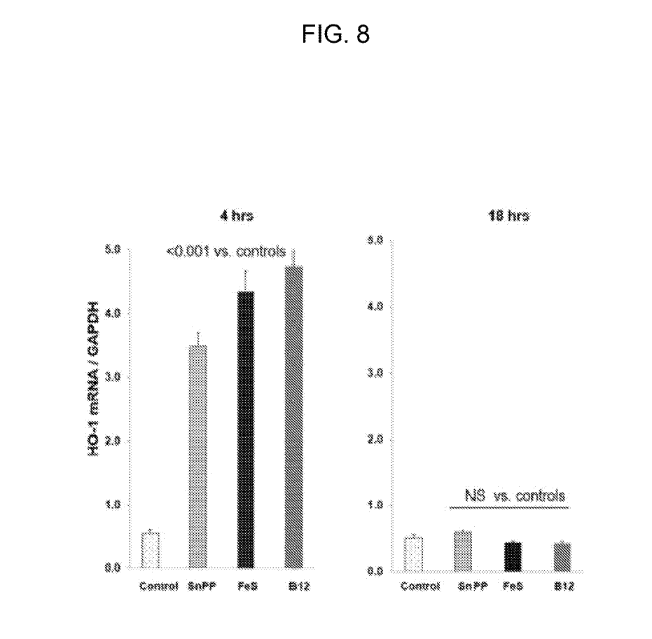

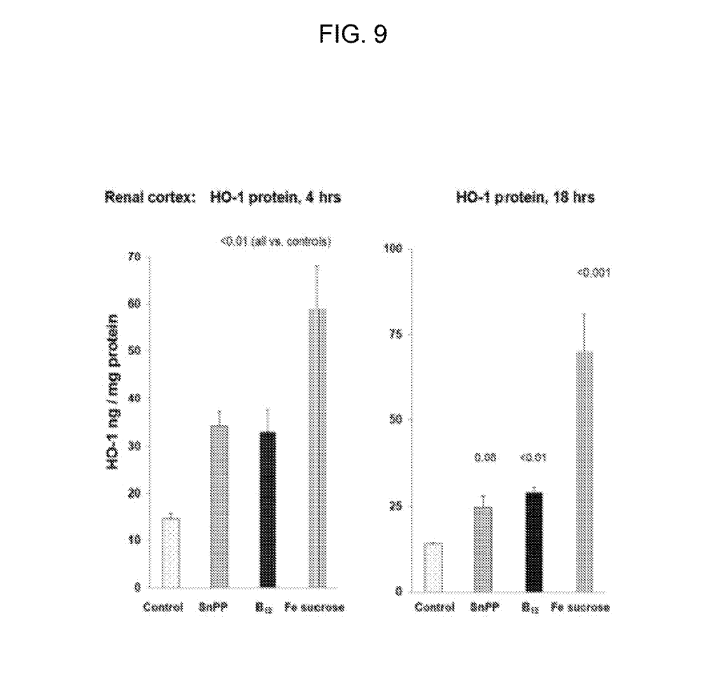

Renal cortical HO-1 mRNA and protein levels. As shown in FIG. 8, each of the agents induced marked and significant increases in HO-1 mRNA, as assessed 4 hr post injection. By 18 hrs, HO-1 mRNA levels returned to normal values. As shown in FIG. 9, a correlate of the 4 hr mRNA increases was a significant increase in HO-1 protein levels. These levels remained elevated at the 18 hr time point, particularly in the case of FeS administration.

### Example 2

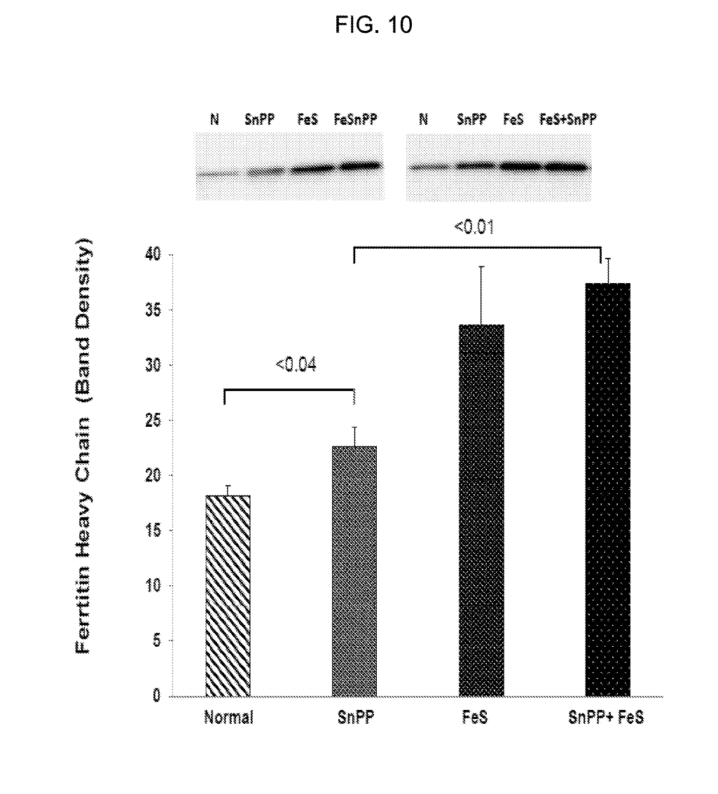

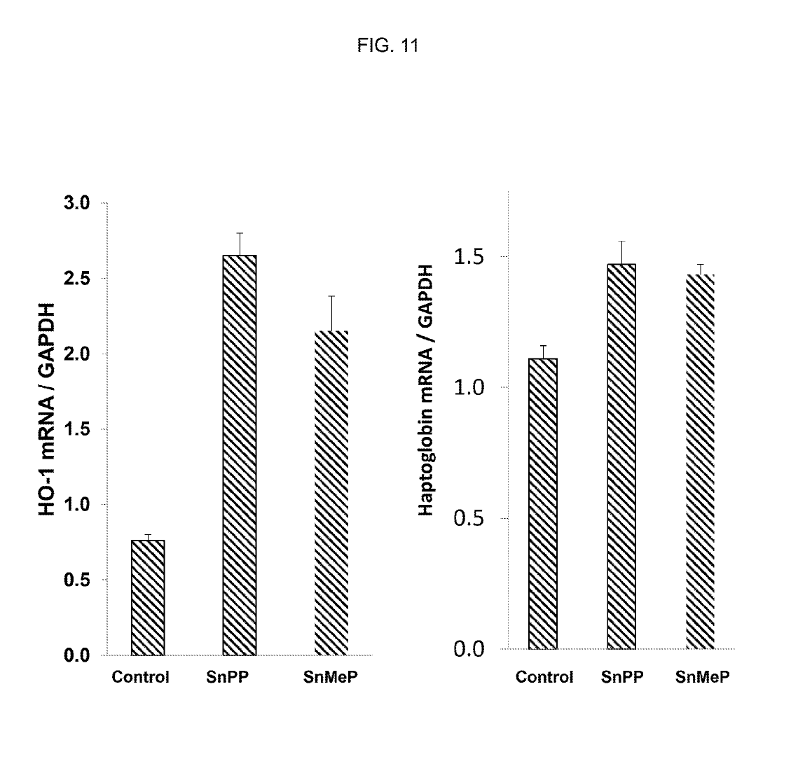

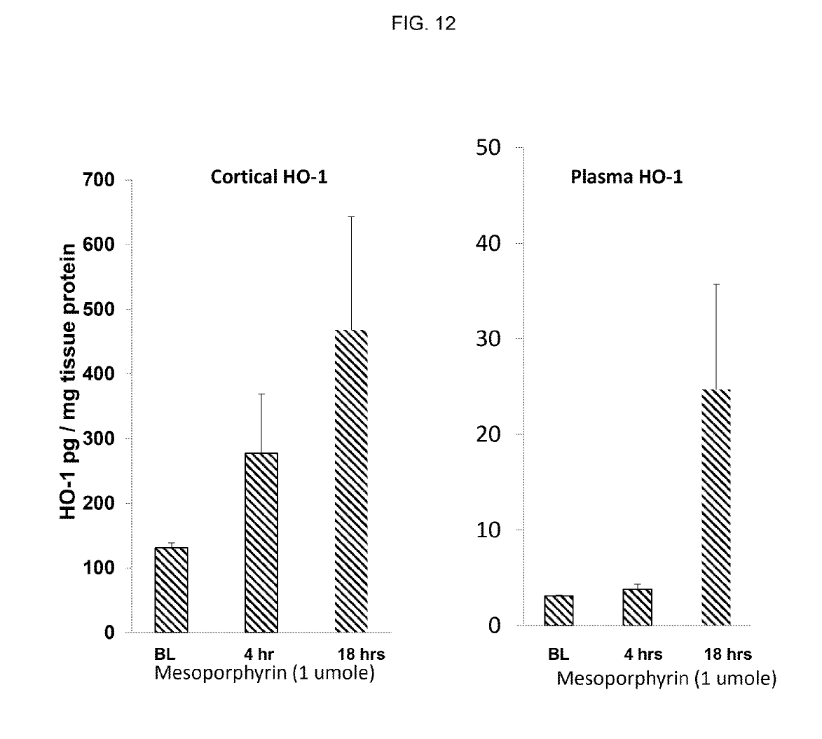

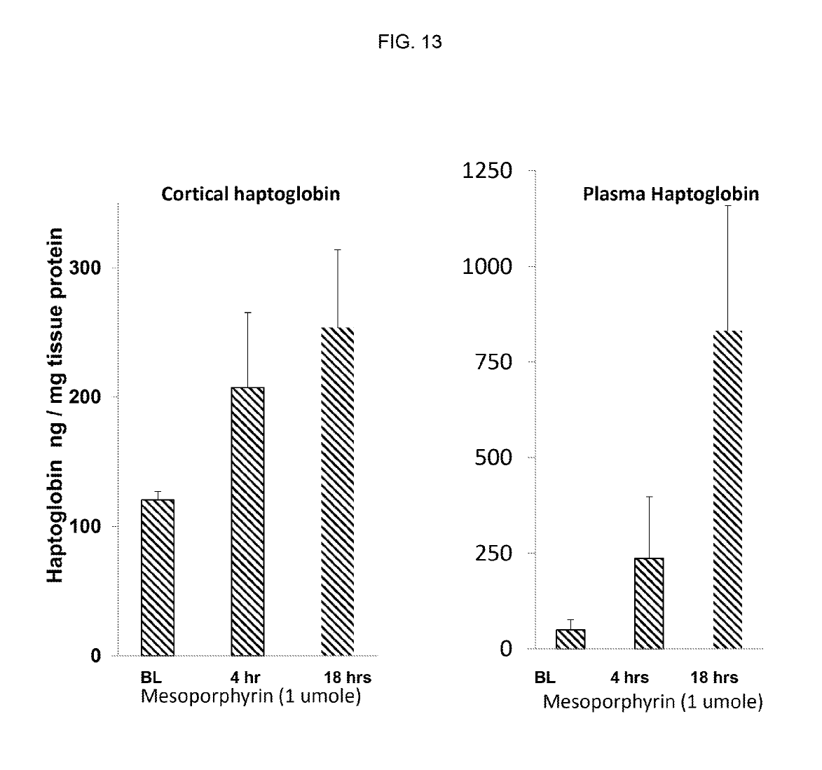

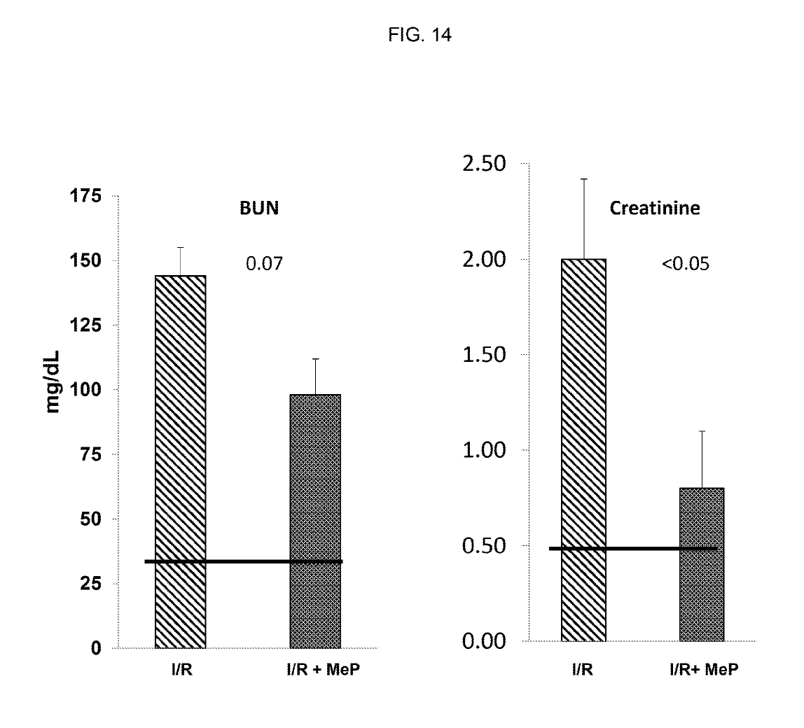

Ferritin heavy (Fhc) expression in renal cortex. HO-1 activity leads to increased Fhc expression. To determine whether SnPP-mediated HO-1 inhibition might impact this result, Fhc was measured by Western blot in renal cortical samples obtained from 10 control mice and 9 mice 18 hr post SnPP treatment. Because Fe sucrose also induces Fhc expression, the potential impact of SnPP treatment on this response was assessed (18 hrs post 1 mg FeS±concomitant SnPP treatment (n, 4 each)). As shown in FIG. 10, SnPP caused a significant 24% increase in Fhc expression over control values, as determined by Western blotting (band densitometry). FeS caused a 1-4% increase in Fhc levels, and this result was fully expressed despite concomitnant SnPP treatment.

### Example 3 Example 10

Introduction: It has been previously documented that inhibition of heme oxygenase 1 (HO-1), using tin protoporphyrin (SnPP), leads to tissue “preconditioning.” Transl Res. 2015 November: 166(5):485-501; and Kidney Int. 2016 90:67-76. This expresses itself as renal protection against diverse forms of renal damage (e.g., ischemia, maleate- or glycerol-induced nephrotoxicity). When an iron containing molecule, most notably nitrited myoglobin or iron sucrose (FeS) is used in conjunction with SnPP, synergistic “preconditioning” results. It has been demonstrated that inhibition of HO-1 confers this protection in part by up-regulating the Nrf2 signal transduction pathway.

Because these protective effects are a result of HO-1 inhibition, rather than being due to a direct drug effect, it has been posited that other HO-1 inhibitors, most notably the mesoporphyrins, (MesP), will recapitulate this protected state.

MesP up-regulates Nrf2 sensitive genes. Mice are injected MesP (0.1-2.5 umoles/mouse) or with a MesP vehicle. At either 4 or 18 hrs, the mice are deeply anesthetized with pentobarbital and the kidneys are removed through a midline abdominal incision. The renal cortices are dissected on ice, extracted for protein and mRNA, and assayed for HO-1 and haptoglobin protein and mRNA levels by ELISA and competitive RT-PCR, respectively. In this regard, both HO-1 and haptoglobin are Nrf2 sensitive genes. An increase in HO-1 and haptoglobin mRNA and protein levels at 4 hrs and 18 hrs post MesP administration, respectively, is measured to confirm MesP, like SnPP, induces Nrf2 signaling.

In additional experiments, MesP is administered in conjunction with 1 mg FeS. An effect on both HO-1 and haptoglobin gene expression are assessed by greater HO-1 and haptoglobin mRNA or protein increases with combination therapy than with either agent alone.

Protection against acute kidney injury. Mice are treated with MesP alone (0.1-20.5 umoles), FeS alone (1 mg), MesP+FeS, or vehicle injection. Eighteen hrs post injections, the mice are anesthetized with pentobarbital. The abdominal cavities are opened. The renal pedicles are identified, and both are occluded with microvascular clamps. Body temperature are maintained at 36-37° C. throughout. Following 22 minutes of bilateral renal ischemia, the clamps are removed, uniform reperfusion are confirmed by the loss of tissue cyanosis, and then the abdominal cavities are closed in two layers with silk suture. Eighteen hrs later, the mice are re-anesthetized, the abdominal cavities are re-opened, and terminal blood samples are obtained from the vena cava. AKI severity are determined by degrees of BUN and plasma creatinine increases. Results are obtained to show that MesP confers protection (lower BUN and plasma creatinine levels than seen in control ischemia mice), and that this protection can be enhanced by concomitant FeS treatment.

### Conclusions

These experiments are performed to demonstrate that HO-1 inhibition (HO-1 inhibitors) can confer a renal protected state, and that this protection can be augmented with concomitant Fe treatment. Moreover, as previously demonstrated, FeS mediated potentiation is a result of Fe, not sucrose effects, and hence this effect can be recapitulated with a variety of Fe containing molecules, (e.g., myoglobin, FeS, hemin injections).

The formulations, kits, and methods disclosed herein are distinguished from “remote preconditioning” whereby one causes ischemia in the legs (e.g., by inflating blood pressure cuffs) to precondition other organs, which has met with only very limited success. In particular embodiments, the compositions, kits and methods disclosed herein can be referred to as “remote pharmacologic preconditioning” (RPR).

As will be understood by one of ordinary skill in the art, each embodiment disclosed herein can comprise, consist essentially of, or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically-significant reduction in the ability of a disclosed composition, kit or method to protect an organ from a scheduled insult.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that can vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the”, and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or example language (e.g., “such as”) provided herein is intended merely to better illuminate the embodiments of the invention and does not pose a limitation on the scope of the embodiments of the invention otherwise claimed. No language in the specification should be construed as indicating any non-claimed element essential to the practice of the embodiments of the invention.

Groupings of alternative elements or embodiments of the invention disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the embodiments of the invention. Of course, variations on these described embodiments will become apparent to those of ordinary skill in the art upon reading the foregoing description. The inventor expects skilled artisans to employ such variations as appropriate, and the inventors intend for the embodiments of the invention to be practiced otherwise than specifically described herein. Accordingly, the embodiments of the invention include all modifications and equivalents of the subject matter recited in the claims appended hereto as permitted by applicable law. Moreover, any combination of the above-described elements in all possible variations thereof is encompassed by the embodiments of the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the embodiments of the invention in more detail than is necessary for the fundamental understanding of the emodiments of the invention, the description taken with the drawings and/or examples making apparent to those skilled in the art how the several forms of the invention can be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3^{rd} Edition or a dictionary known to those of ordinary skill in the art, such as the Oxford Dictionary of Biochemistry and Molecular Biology (Ed. Anthony Smith, Oxford University Press, Oxford, 2004).

In closing, it is to be understood that the embodiments of the invention disclosed herein are illustrative of the principles of the present invention. Other modifications that can be employed are within the scope of the invention. Thus, by way of example, but not of limitation, alternative configurations of the present invention can be utilized in accordance with the teachings herein. Accordingly, the present invention is not limited to that precisely as shown and described.

What is claimed is:

1. A conjugate, or salt thereof, having a structure: 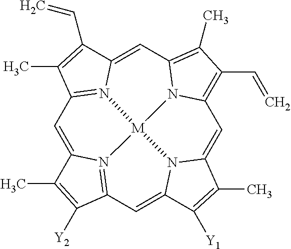
wherein:
M is selected from Sn or Zn;
at least one of Y₁ or Y₂ has a structure 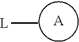
with L being a linking component having from 2 to 10 carbon atoms and A being a Fe-sucrose complex; and
optionally, one of Y₁ or Y₂ is H, an alkyl group having from 1 to 5 carbon atoms, or an alkyl carboxyl group having from 1 to 5 carbon atoms.

2. A conjugate of claim 1 or a salt thereof, wherein L is coupled with A via at least one ester linkage.

3. A conjugate of claim 1 or a salt thereof, wherein the at least one of Y₁ or Y₂ is 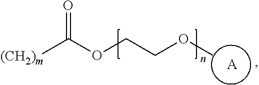
wherein m is from 0 to 5 and n is from 1 to 5.

4. A conjugate of claim 1 or a salt thereof, wherein M is Sn.

5. A conjugate or a salt thereof comprising component A, component B, and a linking component L, wherein component A and component B are linked through L, and wherein:
component A comprises a metal-saccharide complex;
linking component L comprises an aliphatic chain of 1 to 25 carbon atoms;
component B is a compound having a formula 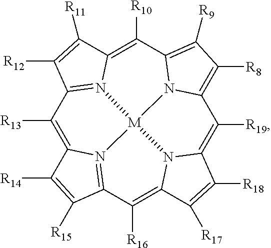
wherein:
M is a metal ion having a charge of ⁺2 or ⁺3;
R₈, R₉, R₁₀, R₁₁, R₁₂, R₁₃, R₁₄, R₁₅, R₁₆, R₁₇, R₁₈, or R₁₉ is independently a hydrogen, a halogen, a hydroxyl group, a nitro group, an amino group, an alkyl group, a cycloalkyl group, an alkenyl group, an alkynyl group, an alkoxy group, an aryl group, an aralkyl group, a carboxyl group, or a heterocyclic group, and R₈, R₉, R₁₀, R₁₁, R₁₂, R₁₃, R₁₄, R₁₅, R₁₆, R₁₇, R₁₈, or R₁₉ is optionally substituted; and
B is linked to L through R₈, R₉, R₁₁, R₁₂, R₁₄, R₁₅, R₁₇, or R₁₈.

6. A conjugate of claim 5 or a salt thereof, wherein the metal of component A is iron.

7. A conjugate of claim 5 or a salt thereof, wherein component A is an iron-sucrose complex.

8. A conjugate of claim 5 or a salt thereof, wherein component B is metal protoporphryin or metal mesoporphyrin.

9. A conjugate of claim 5 or a salt thereof, wherein M of component B is Sn or Zn.

10. A conjugate of claim 5, wherein L is derived from an aliphatic diol having the formula 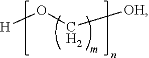
wherein m is an integer from 1 to 5 and n is an integer from 1 to 25.

11. A conjugate of claim 5, wherein L is derived from a polyethylene glycol having the formula 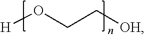
wherein n is an integer from 1 to 25.

12. A composition comprising the conjugate of claim 5 or a salt thereof and a carrier.

13. A pharmaceutical composition comprising the conjugate of claim 5 or a salt thereof and a pharmaceutically acceptable carrier.

14. A pharmaceutical composition of claim 13, wherein the pharmaceutical composition further comprises a heme protein.

15. A pharmaceutical composition of claim 14, wherein the heme protein is hemoglobin or myoglobin.

16. A conjugate, or salt thereof, having a structure: 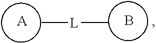
wherein A is a metal-saccharide complex, L comprises one or more linking components having an aliphatic chain comprising less than 25 carbon atoms, and B comprises a compound derived from a cobalaimin or a porphyrin.

17. A conjugate of claim 16 or a salt thereof, wherein the metal of A is Fe.

18. A conjugate of claim 16 or a salt thereof, wherein the saccharide is a disaccharide.

19. A conjugate of claim 16 or a salt thereof, wherein the saccharide is sucrose.

20. A conjugate of claim 16 or a salt thereof, wherein L is derived from a diol.

21. A conjugate of claim 16 or a salt thereof, wherein L is derived from a diacid.

22. A conjugate of claim 16 or a salt thereof, wherein L is derived from an amino acid.

23. A conjugate of claim 16 or a salt thereof, wherein B comprises a compound derived from Sn-protoporphyrin.
